SEQUENCE LISTING (1 sequence)

SEQ ID NO: 1 - DNA, 19 bp, Homo sapiens
  source 1..19: mol_type = genomic DNA
gagtagatcggaaagcatc

(19) United States
(12) Patent Application Publication
(10) Pub. No.: US 2025/0352539 A1
(43) Pub. Date: Nov. 20, 2025

(54) COMBINATION THERAPY FOR TREATING CANCER

(71) Applicant: AstraZeneca AB, Södertälje (SE)

(72) Inventors: Alan Yin Kai LAU, Cambridge (GB); Elisabetta LEO, Cambridge (GB); Giuditta ILLUZZI, Cambridge (GB)

(21) Appl. No.: 18/870,743

(22) PCT Filed: Jun. 14, 2023
(86) PCT No.: PCT/EP2023/066018

§ 371 Date: Dec. 2, 2024

Related U.S. Application Data
(60) Provisional application No. 63/366,407, filed on Jun. 15, 2022.
(60) Provisional application No. 63/386,966, filed on Dec. 12, 2022.

(51) Int. Cl. A61K 31/496; A61K 31/498; A61K 31/5377; A61P 35/00

(52) U.S. Cl. CPC A61K 31/496; A61K 31/498; A61K 31/5377; A61P 35/00

(57) ABSTRACT

The present provides a method of treating ovarian cancer, breast cancer, gastrointestinal cancer, lung cancer, cancer of the brain or prostate cancer in a subject in need thereof, comprising administering to the subject a first amount of a selective PARP1 inhibitor or a pharmaceutically acceptable salt thereof, and a second amount of an ATR inhibitor or a pharmaceutically acceptable salt thereof. Also disclosed are compositions and kits comprising a PARP inhibitor and ATR inhibitor.

The present disclosure relates to methods of treating ovarian cancer, breast cancer, gastrointestinal cancer, lung cancer, cancer of the brain or prostate cancer in a patient in need thereof.

### BACKGROUND

Clinical PARP inhibitors (PARPi) act primarily by “trapping” of PARP1-DNA complexes creating DNA lesions which stall DNA replication fork progression, induce replication stress and activate the Ataxia Telangiectasia and Rad3 Related (ATR)-dependent replication stress response (RSR) pathways to facilitate DNA repair (Cimprich 2008, Forment 2018).

ATR is a serine/threonine protein kinase and multiple small molecule kinase inhibitors of ATR are in clinical development for the treatment on cancer as monotherapy or in combination with targeted agents, chemotherapy/radiotherapy or immune checkpoint blockade (Foote 2015, Barneih 2021).

In particular, ATR inhibition is expected to act synergistically in combination with PARP inhibition leading to increased DNA damage and enhanced anti-tumor activity. Extensive pre-clinical studies of ATR inhibitors e.g. ceralasertib, elimusertib, berzosertib, gartisertib, VE-821, RP-3500 in combination with first generation clinical PARP inhibitors e.g. olaparib, talazoparib, niraparib, rucaparib, have demonstrated greater anti-tumor activity than could be archived with either agent alone.

The clinical use of PARPi in the treatment of epithelial ovarian cancers (EOC) has expanded dramatically. Olaparib, rucaparib, and niraparib were initially approved for use in the recurrence setting as monotherapy (Kim 2015, Balasubramaniam 2017) agnostic of sensitivity to platinum, followed by approval as post-chemotherapy maintenance for platinum sensitive disease (Ison 2018). PARPi are now FDA approved as frontline maintenance. Olaparib obtained FDA approval in 2018 as maintenance following response to frontline platinum-based therapy for patients with germline or somatic BRCA-mutated EOC. In April 2020, niraparib received FDA approval as maintenance following response to frontline platinum regardless of HR status, and the combination of olaparib/bevacizumab received FDA approval in May 2020 as maintenance for patients with HRD EOC.

The combination of PARP inhibitors and certain ATR inhibitors has been demonstrated across a range in PARPi-naïve or PARPi-resistant BRCA1-mutant EOC models (VE-821, Burgess 2020; AZD6738, Kim 2020), breast cancer models (BAY-1895344, Wengner 2020; AZD6738, Wilson 2022; RP-3500, Roulston 2022) and lung cancer models (berzosertib, Gorecki 2020; AZD6738, Lloyd 2020; M4344, Jo 2021). In addition, the combination has shown the ability to overcome mechanisms of innate or acquired PARP inhibitor resistance (Prados-Carvajal 2021), such as through BRCA reversions (Kim 2021), homologous recombination (HR) rewiring (53BP1/Shieldin complex loss) and fork protection pathways which partially restore HR function (Yazinski 2017) or SLFN11-loss (Murai 2016).

The increase in PARPi use is expected to be paralleled by an increasing number of patients who are found to have de novo or acquired resistance to PARPi.

Reports from small scale clinical trials with olaparib and ceralasertib in recurrent platinum-resistant BRCA mutant EOC patients (CAPRI trial, Shah 2021) and BRCA mutant PARP inhibitor resistant HGSOC patients (OLAPCO trial, Madhi 2021) have shown signs of clinical activity.

However, more recently it has been reported that a combination of olaparib and ceralasertib did not improve outcome in previously treated metastatic triple-negative breast cancer versus olaparib alone (Tutt 2022).

While much progress has been made in the treatment of ovarian cancer, breast cancer, gastrointestinal cancer, lung cancer, cancer of the brain or prostate cancer, many of these patients who have such cancers live with an incurable disease. Accordingly, it is important to continue to find new treatments for patients with incurable cancer.

### SUMMARY

In some embodiments, disclosed is a method of treating ovarian cancer, breast cancer, gastrointestinal cancer, lung cancer, cancer of the brain or prostate cancer in a subject in need thereof, comprising administering to the subject a first amount of a selective PARP1 inhibitor or a pharmaceutically acceptable salt thereof, and a second amount of an ATR inhibitor or a pharmaceutically acceptable salt thereof. In the method, the first amount and the second amount together comprise a therapeutically effective amount.

In some embodiments, disclosed is a selective PARP1 inhibitor or a pharmaceutically acceptable salt thereof, for use in the treatment of ovarian cancer, breast cancer, gastrointestinal cancer, lung cancer, cancer of the brain or prostate cancer in a subject, wherein said treatment comprises the separate, sequential or simultaneous administration of i) said selective PARP1 inhibitor or a pharmaceutically acceptable salt thereof, and ii) an ATR inhibitor or a pharmaceutically acceptable salt thereof, to said subject.

In some embodiments, disclosed is an ATR inhibitor or a pharmaceutically acceptable salt thereof, for use in the treatment of ovarian cancer, breast cancer, gastrointestinal cancer, lung cancer, cancer of the brain or prostate cancer in a subject, wherein said treatment comprises the separate, sequential or simultaneous administration of i) said ATR inhibitor or a pharmaceutically acceptable salt thereof, and ii) a selective PARP1 inhibitor or a pharmaceutically acceptable salt thereof, to said subject.

In some embodiments, disclosed is the use of a selective PARP1 inhibitor or a pharmaceutically acceptable salt thereof, in the manufacture of a medicament for use in the treatment of ovarian cancer, breast cancer, gastrointestinal cancer, lung cancer, cancer of the brain or prostate cancer, wherein said treatment comprises the separate, sequential or simultaneous administration of i) said medicament comprising a selective PARP1 inhibitor or a pharmaceutically acceptable salt thereof, and ii) an ATR inhibitor or a pharmaceutically acceptable salt thereof, to said subject.

In some embodiments, disclosed is a pharmaceutical product comprising i) a selective PARP1 inhibitor or a pharmaceutically acceptable salt thereof, and ii) an ATR inhibitor or a pharmaceutically acceptable salt thereof.

In some embodiments, disclosed is a kit comprising: a first pharmaceutical composition comprising a selective PARP1 inhibitor or a pharmaceutically acceptable salt thereof; a second pharmaceutical composition comprising an ATR inhibitor or a pharmaceutically acceptable salt thereof; and instructions for using the first and second pharmaceutical compositions in combination.

The combination of a selective PARP1 inhibitor and an ATR inhibitor may result in fewer side effects or be more effective than current monotherapies or combination therapies. This may result from the selective nature of the PARP1 inhibitor.

### FIGURES

FIG. 1 shows complete loss of expression of 53BP1 protein expression in SUM149PT 53BP1 KO cell pools compared to control SUM149PT 53BP1 WT cell pool (CNTR).

FIG. 2 shows clonogenic growth of SUM149PT CNTR or 53BP1 KO cell pools upon AZD5305 (PARP1Sel=AZD5305) monotherapy treatment.

FIG. 3 shows clonogenic growth of SUM149PT CNTR or 53BP1 KO cell pools upon single AZD5305 (PARP1Sel=AZD5305) dose combined with 5-point concentration response of AZD6738.

### DETAILED DESCRIPTION

### Selective PARP1 Inhibitor

Selective PARP1 inhibitors are compounds which inhibit PARP1 selectively over other members of the PARP family including PARP2, PARP3, PARP5a and PARP6.

Advantageously, the selective PARP1 inhibitor possesses selectivity for PARP1 over PARP2. In an embodiment, the selective PARP1 inhibitor has 10-fold selectivity for PARP1 over PARP2. In a further embodiment, the selective PARP1 inhibitor has 100-fold selectivity for PARP1 over PARP2. In a further embodiment, the selective PARP1 inhibitor has 500-fold selectivity for PARP1 over PARP2.

In some embodiments, the selective PARP1 inhibitor is a compound disclosed in WO2021/013735A1. These compounds are of Formula (I):

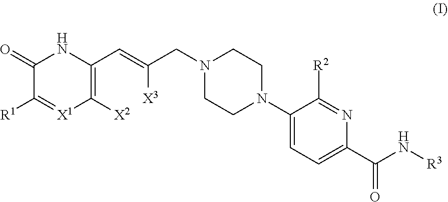

wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹=C(H), and X³ is C(R⁴), and
when X³ is N, then X¹ and X² are both C(H).

Alkyl groups and moieties are straight or branched chain, e.g. C₁₋₈ alkyl, C₁₋₆ alkyl, C₁₋₄ alkyl or C₅₋₆ alkyl. Examples of alkyl groups are methyl, ethyl, n-propyl, iso-propyl, n-butyl, t-butyl, n-pentyl, n-hexyl, n-heptyl and n-octyl, such as methyl or n-hexyl.

Fluoroalkyl groups are alkyl groups in which one or more H atoms is replaced with one or more fluoro atoms, e.g. C₁₋₈ fluoroalkyl, C₁₋₆ fluoroalkyl, C₁₋₄ fluoroalkyl or C₅₋₆ fluoroalkyl. Examples include fluoromethyl (—CH₂F), difluromethyl (—CHF₂), trifluoromethyl (—CF₃), 2,2,2-trifluoroethyl (CF₃CH₂—), 1,1-difluoroethyl (CH₃CHF₂—), 2,2-difluoroethyl (CHF₂CH₂—), and 2-fluoroethyl (CH₂FCH₂—).

Halo means fluoro, chloro, bromo, and iodo. In an embodiment, halo is fluoro or chloro.

In some embodiments, the selective PARP1 inhibitor is “AZD5305”, which refers to a compound with the chemical name 5-{4-[(7-ethyl-6-oxo-5,6-dihydro-1,5-naphthyridin-3-yl)methyl]piperazin-1-yl}-N-methylpyridine-2-carboxamide and structure shown below:

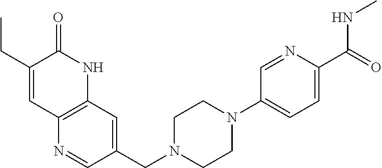 [SMILES: CCc1cc2ncc(CN3CCN(c4ccc(C(=O)NC)nc4)CC3)cc2[nH]c1=O]

AZD5305 is a potent and selective PARP1 inhibitor and PARP1-DNA trapper with excellent in vivo efficacy. AZD5305 is highly selective for PARP1 over other PARP family members, with good secondary pharmacology and physicochemical properties and excellent pharmacokinetics in preclinical species, and with reduced effects on human bone marrow progenitor cells in vitro.

The synthesis of AZD5305 is described in Johannes 2021 and in WO2021/013735, the contents of which are hereby incorporated by reference in their entirety. In some embodiments, a free base AZD5305 is administered to a subject. In some embodiments, a pharmaceutically acceptable salt of AZD5305 is administered to a subject. In some embodiments, crystalline AZD5305 or a pharmaceutically acceptable salt of AZD5305 is administered to a subject.

In some embodiments, the selective PARP1 inhibitor is a compound disclosed in WO2021/260092A1. These compounds are of Formula (II):

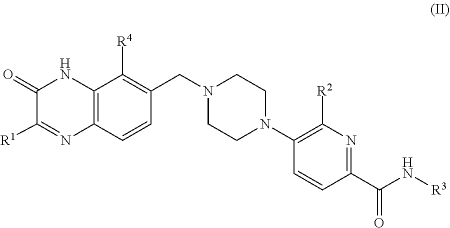

wherein:
R¹ is independently selected from H, C₁₋₄ alkyl, C₁₋₄ fluoroalkyl, and C₁₋₄ alkyloxy;
R² is independently selected from H, halo, C₁₋₄ alkyl, and C₁₋₄ fluoroalkyl; and
R³ is H or C₁₋₄ alkyl;
R⁴ is halo or C₁₋₄ alkyl,
or a pharmaceutically acceptable salt thereof.

Alkyloxy groups are alkyl groups which are connected to the rest of the molecule via an oxygen atom. Examples of suitable C₁₋₄ alkyloxy groups include methoxy, ethoxy, n-propoxy, i-propoxy, n-butoxy, sec-butoxy and t-butoxy.

In some embodiments, the selective PARP1 inhibitor is “AZD9574”, which refers to a compound with the chemical name 6-fluoro-5-[4-[(5-fluoro-2-methyl-3-oxo-4H-quinoxalin-6-yl)methyl]piperazin-1-yl]-N-methylpyridine-2-carboxamide and the structure shown below:

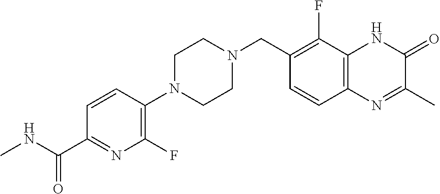

AZD9574 is a blood-brain barrier penetrant PARP1 selective inhibitor. The synthesis of AZD9574 is described in WO2021/260092A1 (example 20), the contents of which are hereby incorporated by reference in their entirety. In some embodiments, a free base AZD9574 is administered to a subject. In some embodiments, a pharmaceutically acceptable salt of AZD9574 is administered to a subject. In some embodiments, crystalline AZD9574 or a pharmaceutically acceptable salt of AZD9574 is administered to a subject.

In some embodiments, the selective PARP1 inhibitor is “AZ14114554”, which refers to a compound with the chemical name 7-((4-(1,5-dimethyl-1H-imidazol-2-yl) piperazin-1-yl)methyl)-3-ethylquinolin-2 (1H)-one and the structure shown below:

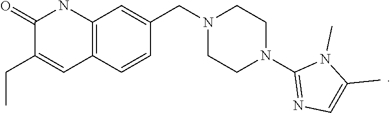 [SMILES: CCc1cc2ccc(CN3CCN(c4ncc(C)n4C)CC3)cc2[nH]c1=O]

The synthesis of AZ14114554 is described in Johannes 2021 (compound 16), the contents of which are hereby incorporated by reference in their entirety. In some embodiments, a free base AZ14114554 is administered to a subject. In some embodiments, a pharmaceutically acceptable salt of AZ14114554 is administered to a subject. In some embodiments, crystalline AZ14114554 or a pharmaceutically acceptable salt of AZ14114554 is administered to a subject.

In some embodiments, the selective PARP1 inhibitor is a compound disclosed in any one of WO2010/133647, WO2011/006794, WO2011/006803, WO2013/014038, WO2013/076090 and WO2014/064149, which are herein incorporated by reference. These selective PARP1 inhibitors have a core which is:

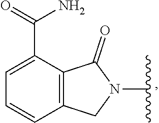 [SMILES: *N1Cc2cccc(C(N)=O)c2C1=O]

and which in some embodiments is:

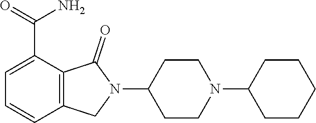 [SMILES: NC(=O)c1cccc2c1C(=O)N(C1CCN(C3CCCCC3)CC1)C2]

Compounds of particular interest are:

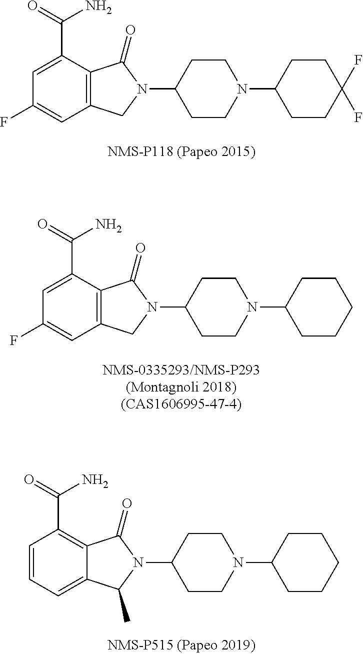 [SMILES: NC(=O)c1cc(F)cc2c1C(=O)N(C1CCN(C3CCC(F)(F)CC3)CC1)C2 ; NC(=O)c1cc(F)cc2c1C(=O)N(C1CCN(C3CCCCC3)CC1)C2 ; C[C@H]1c2cccc(C(N)=O)c2C(=O)N1C1CCN(C2CCCCC2)CC1]

### ATR Inhibitors

The ataxia telangiectasia and Rad3-related (ATR) kinase plays a central role in DNA damage response (DDR) by activating essential signalling pathways of DNA damage repair.

Numerous ATR inhibitors are known, including:
Ceralasertib
Berzosertib
Elimusertib
VE-821
Gartisertib
Camonsertib
AZ20
ATRN-119
ART-0380
IMP-9064
SC-0245
ATG-018
LR-02

These, and other ATR inhibitors, are described in Barnieh 2021. An ATR inhibitor may be suitable for use in the present invention if it meets one or more of the following criteria:
IC₅₀≤100 nM
Selectivity over PI3K>10 fold, for example >100 fold
Selectivity over ATM>10 fold, for example >100 fold
Selectivity over DNA-PK>10 fold, for example >100 fold

“Ceralasertib” refers to a compound with the chemical name 4-{4-[(3R)-3-Methylmorpholin-4-yl]-6-[1-((R)—S-methylsulfonimidoyl)cyclopropyl]pyrimidin-2-yl}-1H-pyrrolo[2,3-b]-pyridine and structure shown below:

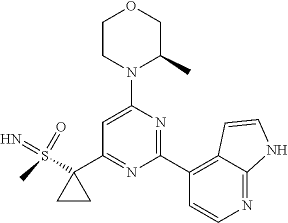 [SMILES: C[C@@H]1COCCN1c1cc(C2([S@](C)(=N)=O)CC2)nc(-c2ccnc3[nH]ccc23)n1]

Ceralasertib (previously known as AZD6738) is an orally available morpholino-pyrimidine-based inhibitor of ataxia telangiectasia and rad3 related (ATR) kinase, with potential antineoplastic activity. Upon oral administration, ceralasertib selectively inhibits ATR activity by blocking the downstream phosphorylation of the serine/threonine protein kinase CHK1. This prevents ATR-mediated signalling, and results in the inhibition of DNA damage checkpoint activation, disruption of DNA damage repair, and the induction of tumor cell apoptosis. In addition, ceralasertib sensitizes tumor cells to chemo- and radiotherapy. ATR, a serine/threonine protein kinase upregulated in a variety of cancer cell types, plays a key role in DNA repair, cell cycle progression and survival; it is activated by DNA damage caused during DNA replication-associated stress.

The synthesis of ceralasertib is described in WO2011/154737 (Example 2.02), WO2020/127208 and Foote 2018, the contents of which are hereby incorporated by reference in their entirety. In some embodiments, a free base ceralasertib is administered to a subject. In some embodiments, a pharmaceutically acceptable salt of ceralasertib is administered to a subject. In some embodiments, crystalline ceralasertib or a pharmaceutically acceptable salt of ceralasertib is administered to a subject.

“Berzosertib” refers to a compound with the chemical name 3-(3-(4-((methylamino)methyl)phenyl)-1,2-oxazol-5-yl)-5-(4-(propane-2-sulfonyl)phenyl)pyrazin-2-amine and structure shown below:

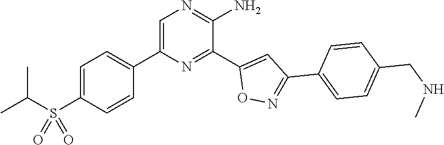 [SMILES: CNCc1ccc(-c2cc(-c3nc(-c4ccc(S(=O)(=O)C(C)C)cc4)cnc3N)on2)cc1]

It was previously known as M-6620 and VX-970. It is a potent ATR inhibitor and less-potent inhibitor of ATM serine/threonine kinase (ATM).

The synthesis of berzosertib is described in WO2010/071837 (Example 57a—compound IIA-7), and Knegtel 2019, the contents of which are hereby incorporated by reference in their entirety. In some embodiments, a free base berzosertib is administered to a subject. In some embodiments, a pharmaceutically acceptable salt of berzosertib is administered to a subject. In some embodiments, crystalline berzosertib or a pharmaceutically acceptable salt of berzosertib is administered to a subject.

“Elimusertib” refers to a compound with the chemical name 2-[(3R)-3-methylmorpholin-4-yl]-4-(1-methyl-1H-pyrazol-5-yl)-8-(1H-pyrazol-5-yl)-1,7-naphthyridine and structure shown below:

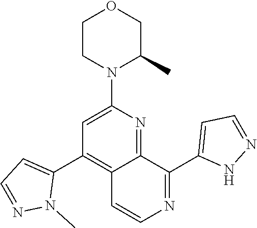 [SMILES: C[C@@H]1COCCN1c1cc(-c2ccnn2C)c2ccnc(-c3ccn[nH]3)c2n1]

The synthesis of elimusertib (previously known as BAY-1895344) is described in WO2016/020320 (Example 111), the contents of which are hereby incorporated by reference in their entirety. In some embodiments, a free base elimusertib is administered to a subject. In some embodiments, a pharmaceutically acceptable salt of elimusertib is administered to a subject. In some embodiments, crystalline elimusertib or a pharmaceutically acceptable salt of elimusertib is administered to a subject.

“VE-821” refers to a compound with the chemical name 3-amino-N,6-diphenylpyrazine-2-carboxamide and the structure shown below:

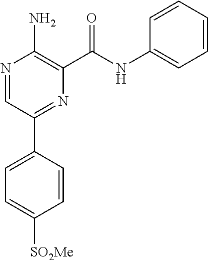 [SMILES: CS(=O)(=O)c1ccc(-c2cnc(N)c(C(=O)Nc3ccccc3)n2)cc1]

The synthesis of VE-821 is described in Charrier 2011 (compound 6), the contents of which are hereby incorporated by reference in their entirety. In some embodiments, a free base VE-821 is administered to a subject. In some embodiments, a pharmaceutically acceptable salt of VE-821 is administered to a subject. In some embodiments, crystalline VE-821 or a pharmaceutically acceptable salt of VE-821 is administered to a subject.

“Gartisertib” refers to a compound with the chemical name 2-amino-6-fluoro-N-[5-fluoro-4-(4-{[4-(3-oxetanyl)-1-piperazinyl]carbonyl}-1-piperidinyl)-3-pyridinyl]pyrazolo[1,5-a]pyrimidine-3-carboxamide and the structure shown below:

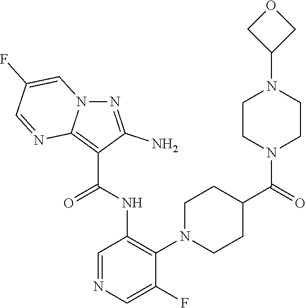 [SMILES: Nc1nn2cc(F)cnc2c1C(=O)Nc1cncc(F)c1N1CCC(C(=O)N2CCN(C3COC3)CC2)CC1]

Gartisertib (previously known as M4344 and VX-803) is described in Zenke 2019 and Jo 2021, the contents of which are hereby incorporated by reference in their entirety. In some embodiments, a free base gartisertib is administered to a subject. In some embodiments, a pharmaceutically acceptable salt of gartisertib is administered to a subject. In some embodiments, crystalline gartisertib or a pharmaceutically acceptable salt of gartisertib is administered to a subject.

“Camonsertib” refers to a compound with the chemical name (1R,3R,5S)-3-6-[(3R)-3-methylmorpholin-4-yl]-1-(1H-pyrazol-3-yl)-1H-pyrazolo[3,4-b]pyridin-4-yl-8-oxabicyclo[3.2.1]octan-3-ol and the structure shown below:

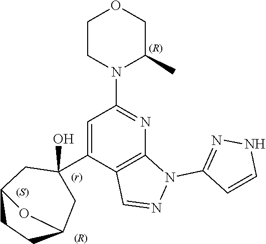

Camonsertib (previously known as RP-3500) is described in Roulston 2022, the contents of which are hereby incorporated by reference in their entirety. In some embodiments, a free base camonsertib is administered to a subject. In some embodiments, a pharmaceutically acceptable salt of camonsertib is administered to a subject. In some embodiments, crystalline camonsertib or a pharmaceutically acceptable salt of camonsertib is administered to a subject.

“AZ20” refers to a compound with the chemical name 4-{4-[(3R)-3-Methylmorpholin-4-yl]-6-[1-(methylsulfonyl)cyclopropyl]pyrimidin-2-yl}-1H-indole and the structure shown below:

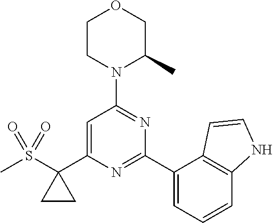 [SMILES: C[C@@H]1COCCN1c1cc(C2(S(C)(=O)=O)CC2)nc(-c2cccc3[nH]ccc23)n1]

AZ20 is described in Foote 2013, the contents of which are hereby incorporated by reference in their entirety. In some embodiments, a free base AZ20 is administered to a subject. In some embodiments, a pharmaceutically acceptable salt of AZ20 is administered to a subject. In some embodiments, crystalline AZ20 or a pharmaceutically acceptable salt of AZ20 is administered to a subject.

“ATRN-119” refers to a compound from ATRIN, which is about to clinical trials (NCT04905914), and which is described in WO2016/061097. It is also discussed in, for example, Gilad 2020 and George 2018.

“ART-0380” refers to a compound from Artios, which is in Phase 1 clinical trials (NCT04657068). It is also discussed in, for example, in Patel 2022.

“IMP-9064” refers to a compound from IMPACT, which is in clinical trials (NCT05269316; CXHL2101780).

“SC-0245” refers to a compound from Wuxi Apptec, which is in clinical trials (CTR20210769), and which is described in WO2021/023272. It is also discussed, for example, in Wang 2020.

“ATG-018” refers to a compound from Antegene, which is in clinical trials (NCT05338346). It is also discussed in, for example, in Yuwen 2022.

“LR-02” refers to a compound from Laevoroc Oncology, which is discussed in, for example, Koul 2021.

In some embodiments, the selective PARP1 inhibitor is either AZD5305 or AZD9574 and the ATR inhibitor is ceralasertib. In some of these embodiments, the selective PARP1 inhibitor is AZD5305 and the ATR inhibitor is ceralasertib. In other of these embodiments, the selective PARP1 inhibitor is AZD9574 and the ATR inhibitor is ceralasertib.

The language “pharmaceutical composition” includes compositions comprising an active ingredient and a pharmaceutically acceptable excipient, carrier or diluent, wherein the active ingredient is a selective PARP1 inhibitor or a pharmaceutically acceptable salt thereof, or an ATR inhibitor or a pharmaceutically acceptable salt thereof. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art.

In some embodiments, the pharmaceutical compositions are in solid dosage forms, such as capsules, tablets, granules, powders or sachets. In some embodiments, the pharmaceutical compositions are in the form of a sterile injectable solution in one or more aqueous or non-aqueous non-toxic parenterally acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “treat,” “treating” and “treatment” includes the reduction or inhibition of enzyme or protein activity related to PARP-1, ATR or ovarian cancer, breast cancer, gastrointestinal cancer, lung cancer, cancer of the brain or prostate cancer in a subject, amelioration of one or more symptoms of ovarian cancer, breast cancer, gastrointestinal cancer, lung cancer, cancer of the brain or prostate cancer in a subject, or the slowing or delaying of progression of ovarian cancer, breast cancer, gastrointestinal cancer, lung cancer, cancer of the brain or prostate cancer in a subject. The language “treat,” “treating” and “treatment” also includes the reduction or inhibition of the growth of a tumor or proliferation of cancerous cells in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from ovarian cancer, breast cancer, gastrointestinal cancer, lung cancer, cancer of the brain or prostate cancer.

The language “therapeutically effective amount” includes that amount of the selective PARP1 inhibitor and that amount of the ATR inhibitor which together will elicit a biological or medical response in a subject, for example, the reduction or inhibition of enzyme or protein activity related to PARP1, ATR, or cancer; amelioration of symptoms of ovarian cancer, breast cancer, gastrointestinal cancer, lung cancer, cancer of the brain or prostate cancer; or the slowing or delaying of progression of ovarian cancer, breast cancer, gastrointestinal cancer, lung cancer, cancer of the brain or prostate cancer. In some embodiments, the language “therapeutically effective amount” includes the amount of the selective PARP1 inhibitor and the ATR inhibitor together that is effective to at least partially alleviate, inhibit, and/or ameliorate ovarian cancer, breast cancer, gastrointestinal cancer, lung cancer, cancer of the brain or prostate cancer or inhibit PARP1 or ATR, and/or reduce or inhibit the growth of a tumor or proliferation of cancerous cells in a subject.

Without wishing to be bound by theory, the combination of a selective PARP1 inhibitor with an ATR inhibitor may provide a more favourable tolerability profile, higher drug exposures and more durable target inhibition leading to greater anti-tumor efficacy and combination options than combinations of first-generation PARP inhibitors with an ATR inhibitor.

In some embodiments, disclosed is a method of treating ovarian cancer, breast cancer, gastrointestinal cancer, lung cancer, cancer of the brain or prostate cancer in a subject in need thereof, comprising administering to the subject a first amount of a selective PARP1 inhibitor or a pharmaceutically acceptable salt thereof, and a second amount of an ATR inhibitor or a pharmaceutically acceptable salt thereof. In the method, the first amount and the second amount together comprise a therapeutically effective amount.

In some embodiments, disclosed is a selective PARP1 inhibitor, or a pharmaceutically acceptable salt thereof, for use in the treatment of ovarian cancer, breast cancer, gastrointestinal cancer, lung cancer, cancer of the brain or prostate cancer in a subject, wherein said treatment comprises the separate, sequential or simultaneous administration of i) said selective PARP1 inhibitor, or a pharmaceutically acceptable salt thereof, and ii) an ATR inhibitor, or a pharmaceutically acceptable salt thereof, to said subject.

In some embodiments, disclosed is an ATR inhibitor, or a pharmaceutically acceptable salt thereof, for use in the treatment of ovarian cancer, breast cancer, gastrointestinal cancer, lung cancer, cancer of the brain or prostate cancer in a subject, wherein said treatment comprises the separate, sequential or simultaneous administration of i) said ATR inhibitor, or a pharmaceutically acceptable salt thereof, and ii) a selective PARP1 inhibitor, or a pharmaceutically acceptable salt thereof, to said subject.

In some embodiments, disclosed is the use of a selective PARP1 inhibitor, or a pharmaceutically acceptable salt thereof, in the manufacture of a medicament for use in the treatment of ovarian cancer, breast cancer, gastrointestinal cancer, lung cancer, cancer of the brain or prostate cancer in a subject, wherein said treatment comprises the separate, sequential or simultaneous administration of i) said medicament comprising a selective PARP1 inhibitor, or a pharmaceutically acceptable salt thereof, and ii) ATR inhibitor, or a pharmaceutically acceptable salt thereof, to said subject.

In some embodiments, a selective PARP1 inhibitor or a pharmaceutically acceptable salt thereof and an ATR inhibitor or a pharmaceutically acceptable salt thereof are administered separately, sequentially or simultaneously in a treatment cycle. In some embodiments, a selective PARP1 inhibitor or a pharmaceutically acceptable salt thereof is continuously administered in the treatment cycle and an ATR inhibitor or a pharmaceutically acceptable salt is also continuously administered in the treatment cycle.

The term “continuous” or “continuously” refers to administration of a therapeutic agent, e.g. the selective PARP1 inhibitor, at regular intervals without stopping or interruption, i.e., no void day. By “void day”, it is meant a day when a therapeutic agent is not administered.

The term “intermittent” or “intermittently” as used herein means stopping and starting administration of a therapeutic agent at either regular or irregular intervals in a treatment cycle. For intermittent administration, there is at least one void day in the treatment cycle.

A “cycle”, “treatment cycle” or “dosing schedule”, as used herein, refers to a period of combination treatment that is repeated on a regular schedule. For example, the treatment can be given for one week, two weeks, or three weeks wherein the selective PARP1 inhibitor and an ATR inhibitor are administered in a coordinated fashion. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months. In some embodiments, the period of rest, i.e., void day(s), in a treatment cycle is about 1 day to about 1 month. In some embodiments, the period of rest in a treatment cycle is about 1 day, about 3 days, about 5 days, about 1 week, about 2 weeks, or about 3 weeks.

In some embodiments, the selective PARP1 inhibitor or a pharmaceutically acceptable salt thereof and an ATR inhibitor or a pharmaceutically acceptable salt thereof are administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

### AZD5305 Dosing

In some embodiments, AZD5305 or a pharmaceutically acceptable salt thereof is administered for 28 days in a 28-day treatment cycle. In some embodiments, AZD5305 or a pharmaceutically acceptable salt thereof is dosed in an intermitted schedule.

In some embodiments, AZD5305 or a pharmaceutically acceptable salt thereof is administered orally. In some embodiments, AZD5305 or a pharmaceutically acceptable salt thereof is in tablet dosage form. In some embodiments, AZD5305 is administered in a dose of up to about 60 mg (for example, up to 0.5 mg, up to 1 mg, up to about 2.5 mg, up to about 5 mg, up to about 10 mg, up to about 15 mg, up to about 20 mg, up to about 25 mg, up to about 30 mg, up to about 35 mg, up to about 40 mg, up to about 45 mg, up to about 50 mg, up to about 55 mg, or up to about 60 mg AZD5305) per day. In some embodiments, AZD5305 is administered once a day (QD). In some embodiments, AZD5305 is administered in a dose of about 0.5 mg QD, about 1 mg QD, about 2.5 mg QD, about 5 mg QD, about 10 mg QD, about 15 mg QD, about 20 mg QD, about 25 mg QD, about 30 mg QD, about 35 mg QD, about 40 mg QD, about 45 mg QD, about 50 mg QD, about 55 mg QD or about 60 mg QD.

In some further embodiments, AZD5305 is administered in a dose of up to about 140 mg (for example, up to about 80 mg, up to about 90 mg, up to about 100 mg, up to about 110 mg, up to about 120 mg, or up to about 140 mg AZD5305) per day. In some further embodiments, AZD5305 is administered in a dose of about 80 mg QD, about 90 mg QD, about 100 mg QD, about 110 mg QD, about 120 mg QD, or about 140 mg QD.

### PARP1 Selective Inhibitor Dosing

In some embodiments, the PARP1 selective inhibitor may be dosed in the same manner as AZD5305 described above.

### ATR Inhibitor Dosing

In some embodiments, the ATR inhibitor is dosed in an intermittent schedule, such as 7 or 14 consecutive days in a 28-day treatment cycle, i.e. there is a three or two week rest period, or 3 consecutive days in a 7-day or 14-day treatment cycle, i.e. there is a 4 day or 11 day rest period.

### Ceralasertib Dosing

In some embodiments, ceralasertib or a pharmaceutically acceptable salt thereof is administered for 7 or 14 consecutive days in a 28-day treatment cycle, i.e. there is a three or two week rest period.

In some embodiments, ceralasertib or a pharmaceutically acceptable salt thereof is administered orally. In some embodiments, ceralasertib or a pharmaceutically acceptable salt thereof is in tablet dosage form. In some embodiments, ceralasertib or a pharmaceutically acceptable salt thereof is administered in a dose of up to about 320 mg (for example, up to about 120 mg, up to about 140 mg, up to about 160 mg, up to about 180 mg, up to about 200 mg, up to about 220 mg, up to about 240 mg, up to about 280 mg, or up to about 320 mg ceralasertib) orally per day. In some embodiments, ceralasertib is administered twice per day (BID). In some embodiments, ceralasertib is administered in a dose of about 60 mg BID, about 80 mg BID, about 100 mg BID, about 120 mg BID, about 140 mg BID, or about 160 mg BID. In some embodiments, the 160 mg dose comprise 80 mg or 160 mg tablets.

### Elimusertib Dosing

In some embodiment, elimusertib or a pharmaceutically acceptable salt thereof is administered is administered for 3 consecutive days in a 7-day treatment cycle or for 3 consecutive days in a 14-day treatment cycle.

In some embodiments, elimusertib or a pharmaceutically acceptable salt thereof is administered orally. In some embodiments, elimusertib or a pharmaceutically acceptable salt thereof is in tablet dosage form. In some embodiments, elimusertib or a pharmaceutically acceptable salt thereof is administered in a dose of up to about 80 mg (for example, up to about 20 mg, up to about 40 mg, up to about 60 mg, or up to about 80 mg orally per day).

### Camonsertib Dosing

In some embodiment, camonsertib or a pharmaceutically acceptable salt thereof is administered is administered for 3 consecutive days in a 7-day treatment cycle.

In some embodiments, camonsertib or a pharmaceutically acceptable salt thereof is administered orally. In some embodiments, camonsertib or a pharmaceutically acceptable salt thereof is in tablet dosage form. In some embodiments, camonsertib or a pharmaceutically acceptable salt thereof is administered in a dose of up to about 200 mg (for example, up to about 40 mg, up to about 60 mg, up to about 80 mg, up to about 100 mg, up to about 120 mg, up to about 140 mg, up to about 160 mg, up to about 180 mg, or up to about 200 mg orally per day).

### Combination Dosing

In some embodiments, AZD5305 and ceralasertib are taken separately, where a dose of

AZD5305 is taken on an empty stomach, with no food two hours before, and a dose of ceralasertib is taken at the same time as AZD5305 and with a glass (about 250 ml) of water.

In some embodiments, AZD5305 is administered in a dose of about 2.5 mg QD and Ceralasertib is administered in a dose of about 120 mg BID.

In some embodiments, AZD5305 is administered in a dose of about 2.5 mg QD and Ceralasertib is administered in a dose of about 160 mg BID.

In some embodiments, AZD5305 is administered in a dose of about 5 mg QD and Ceralasertib is administered in a dose of about 160 mg BID.

In some embodiments, disclosed is a pharmaceutical product comprising i) a selective PARP1 inhibitor or a pharmaceutically acceptable salt thereof, and ii) an ATR inhibitor or a pharmaceutically acceptable salt thereof. In some embodiments, the selective PARP1 inhibitor or a pharmaceutically acceptable salt thereof, and ATR inhibitor or a pharmaceutically acceptable salt thereof are present in a single dosage form. In some embodiments, selective PARP1 inhibitor or a pharmaceutically acceptable salt thereof, and ATR inhibitor or a pharmaceutically acceptable salt thereof are present separate dosage forms.

In some embodiments, disclosed is a kit comprising: a first pharmaceutical composition comprising a selective PARP1 inhibitor, or a pharmaceutically acceptable salt thereof; a second pharmaceutical composition comprising an ATR inhibitor, or a pharmaceutically acceptable salt thereof; and instructions for using the first and second pharmaceutical compositions in combination.

### Cancers

In some embodiments, the cancer is ovarian cancer. In certain embodiments, the cancer is advanced epithelial ovarian cancer. In certain embodiments, the cancer is high-grade serous ovarian cancer. In certain embodiments, the cancer is high-grade endometrioid ovarian cancer. In certain embodiments, the cancer is epithelial ovarian cancer comprising a gBRCA1 or a gBRCA2 mutation, or a mutation in any one of ATM, BRIP1, BARD1, CDK12, CHEK1, CHEK2, FANCL, PALB2, PPP2R2A, RAD51B, RAD51C, RAD51D, and RAD54L. In certain embodiments, the ovarian cancer is platinum-sensitive relapsed ovarian cancer, following treatment with a PARP inhibitor. In some of these embodiments, there is no intervening chemotherapy following treatment with the PARP inhibitor.

In some embodiments, the cancer is breast cancer. In some embodiments, the cancer is deleterious or suspected deleterious gBRCAm, HER2-negative metastatic breast cancer. In some embodiments, the cancer is deleterious or suspected deleterious gBRCAm, HER2-negative metastatic breast cancer and has been treated with chemotherapy in the neoadjuvant, adjuvant, or metastatic setting. In some embodiments, the cancer is deleterious or suspected deleterious gBRCAm, HER2-negative, hormone receptor (HR)-positive breast cancer and has been treated with chemotherapy in the neoadjuvant, adjuvant or metastatic setting and has been treated with a prior endocrine therapy or been considered inappropriate for endocrine therapy. In certain embodiments, the breast cancer is triple negative breast cancer.

In some embodiments, the cancer is gastrointestinal cancer. In some of these embodiments, the gastrointestinal cancer is gastric cancer. In some of these embodiments, the gastrointestinal cancer is colorectal cancer. In some of these embodiments, the gastrointestinal cancer is stomach cancer. In some of these embodiments, the gastrointestinal cancer is liver cancer. In some of these embodiments, the gastrointestinal cancer is gallbladder cancer. In some of these embodiments, the gastrointestinal cancer is anal cancer. In some embodiments, the gastrointestinal cancer is pancreatic adenocarcinoma. In some embodiments, the gastrointestinal cancer is deleterious or suspected deleterious gBRCAm pancreatic adenocarcinoma. In some embodiments, the gastrointestinal cancer is deleterious or suspected deleterious gBRCAm pancreatic adenocarcinoma and the disease has not progressed on at least 16 weeks for a first-line platinum-based chemotherapy regimen.

In some embodiments, the cancer is lung cancer. In some of these embodiments, the lung cancer is small cell lung cancer. In further of these embodiments, the lung cancer is non-small cell lung cancer.

In some embodiments, the cancer is cancer of the brain. In some of these embodiments, the cancer of the brain is glioma. In further of these embodiments, the cancer of the brain is glioblastoma. In some embodiments, the cancer of the brain is a metastatic cancer arising from a tumour elsewhere in the body such as breast, ovary, pancreas, prostate, haematological, gastrointestinal such as gastric and colorectal, or lung cancer such as small cell or non-small cell lung cancer.

In some embodiments, the cancer is platinum-resistant.

In some embodiments, the prostate cancer is metastatic prostate cancer, hormone sensitive prostate cancer (HSPC) or castrate resistant prostate cancer (CRPC). In some embodiments, the metastatic prostate cancer may be metastatic hormone sensitive prostate cancer (mHSPC) or metastatic castrate resistant prostate cancer (mCRPC). Metastatic prostate cancer refers to prostate cancer which has spread or metasised to another part of the body.

Hormone sensitive prostate cancer (HSPC) refers to prostate cancer whose growth is inhibited by a decrease in androgen levels or by inhibiting androgen action.

Castrate resistant prostate cancer (CRPC) refers to prostate cancer which continues to grow even when androgen levels in the body are extremely low or undetectable.

Metastatic hormone sensitive prostate cancer (mHSPC) refers to prostate cancer which has spread or metasised to another part of the body, and whose growth is inhibited by a decrease in androgen levels or by inhibiting androgen action.

Metastatic castrate resistant prostate cancer (mCRPC) refers to prostate cancer which has spread or metasised to another part of the body, and which continues to grow even when androgen levels in the body are extremely low or undetectable.

In some embodiments where prostate cancer is being treating, treatment with a luteinising hormone-releasing hormone (LHRH) agonist or antagonist may be administered concurrently, especially if the patient has not undergone an orchidectomy or a subcapsular orchidectomy. LHRH agonists include leuprolide/leuprorelin, goserelin, triptorelin, histrelin, and buserelin. LHRH antagonists include degarelix, relugolix, bicalutamide, flutamide and cyproterone acetate. Such additional treatments may be dosed at the current standard of care.

In some embodiments, the cancer treated may be deficient in Homologous Recombination (HR) dependent DNA DSB repair activity. The HR dependent DNA DSB repair pathway repairs double-strand breaks (DSBs) in DNA via homologous mechanisms to reform a continuous DNA helix (Khanna and Jackson 2001). The components of the HR dependent DNA DSB repair pathway include, but are not limited to, ATM (NM_000051), RAD51 (NM_002875), RAD51L1 (NM_002877), RAD51C (NM_002876), RAD51L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485). Other proteins involved in the HR dependent DNA DSB repair pathway include regulatory factors such as EMSY (Hughes-Davies 2003). HR components are also described in Wood 2001.

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a prostate cancer which is deficient in HR dependent DNA DSB repair. Components of the HR dependent DNA DSB repair pathway are well characterised in the art (see for example, Wood 2001) and include the components listed above.

In some embodiments, the cancer cells may have a BRCA1 and/or a BRCA2 deficient phenotype i.e. BRCA1 and/or BRCA2 activity is reduced or abolished in the prostate cancer cells. Cancer cells with this phenotype may be deficient in BRCA1 and/or BRCA2, i.e. expression and/or activity of BRCA1 and/or BRCA2 may be reduced or abolished in the prostate cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies 2003).

BRCA1 and BRCA2 are known tumour suppressors whose wild-type alleles are frequently lost in tumours of heterozygous carriers (Jasin 2002; Tutt 2002).

In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen and Nder 1992; Chappuis and Foulkes 2002; Janatová 2003; Jancárková 2003). Determination of amplification of the BRCA2 binding factor EMSY is described in Hughes-Davies 2003.

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

In some embodiments, the cancer treated may not be deficient in Homologous Recombination (HR) dependent DNA DSB repair activity.

In some embodiments, the cancer treatment may be resistant to treatment with a PARP inhibitor alone. Resistance to a PARP inhibitor alone may be characterised by disease progression when treated with a PARP inhibitor alone.

In some of these embodiments, the patient will have demonstrated a clinical benefit of treatment with a PARP inhibitor by an initial response to PARP inhibitor treatment or clinical benefit from PARP inhibitor treatment as maintenance therapy followed by disease progression. Clinical benefit for maintenance is defined as:
prior maintenance with PARP inhibitor after 1st line chemotherapy of at least 12 months or
prior maintenance with PARP inhibitor after >1 line of chemotherapy for minimum of 6 months.

In some of these embodiments, the resistance may be caused by:
(a) Epithelial-mesenchymal transition (EMT);
(b) Loss of expression of the Schlafen 11 (SLFN11) gene;
(c) Overexpression of the ATP-binding cassette (ABC) drug efflux transporter Pglycoprotein ABCB1, also known as MDR1;
(d) poly(ADP-ribose) glycohydrolase (PARG) loss;
(e) PARP1 mutation;
(f) BRCA/HRR-Dependent Mechanisms.

PARP inhibitor resistance is discussed in Prados Carvajal 2022.

### EXAMPLES

The compounds of the application will now be further explained by reference to the following non-limiting examples.

### HSA Synergy Score

The Highest Single Agent (HSA) model is used to determine a combination's synergy score (and is based simply on the intuition that if a combination's effect exceeds the effect level of each of its constituents, there must be some combination interaction). Mathematically, the HSA model describes simple superposition of the single agent curves:

$${I}_{HSA} ( {C}_{X} , {C}_{Y} ) = max ( {I}_{X} , {I}_{Y} )$$

where C_{X,Y} are the concentrations of the X and Y compound, and I_{X} and I_{Y} are inhibitions of the single agents at C_{X,Y}. It is also useful to calculate a volume score (HSA Volume) between the data and the HSA surface to characterise the overall strength of combination effects. Empirically derived combination matrices are compared to their respective HSA additivity models constructed from experimentally collected single agent dose response curves. Summation of this excess additivity across the dose response matrix is referred to HSA Volume. Positive HSA Volume suggests potential synergy, while negative HSA Volume suggests potential antagonism.

### Combination Index (CI)

Potency shifting can also be scored using combination index (CI). For a chosen iso-effect level (ICut), CI is calculated as:

$$CI = ( {C}_{X} / {EC}_{X} ) + ( {C}_{Y} / {EC}_{Y} )$$

where (C_{X}/EC_{X}) for a particular data point is the ratio of the X compound's measured concentration to its effective concentration at the chosen effect level. The CI is a rough estimate of how much drug was needed in combination relative to the single agent doses required to achieve the chosen effect level. CI values in the range of 0.5-0.7 are typical for in vitro measurements of current clinical combinations. The CI error (σCI) is calculated using standard error propagation through the CI calculation based on the isobologram errors.

### Example 1—In Vitro Combination Assay

This combination screen was a 10-day assay using Cell Titre Glo as the readout for viability.

The assay was performed in 384 well plates, with 1 cell line and 4 drug-drug combinations per plate dosed in a 6×6 matrix. A day zero reading was measured to determine growth inhibition. GenedataScreener was used to input the raw values for every well, and the software was programmed to normalise the values to the day zero and DMSO control values.

The CellTiter-Glo® Luminescent Cell Viability Assay is a homogeneous method to determine the number of viable cells in culture based on quantitation of the ATP present, which signals the presence of metabolically active cells. It relies on the properties of a proprietary thermostable luciferase (Ultra-Glo™ Recombinant Luciferase), which generates a stable “glow-type” luminescent signal and improves performance across a wide range of assay conditions.

The following information was reported: Synergy scores (HSA); combination index values; AC₅₀ (half-maximal activity concentration) monotherapy values.

### Breast Cell Lines (AZD5305 & AZD6738)

| | | Synergy | AZD5305 | AZD6738 |
|---|---|---|---|---|
| | Maximum | Score | mono | Mono |
| Cell Line | CI | (HSA) | AC₅₀ (M) | AC₅₀ (M) |
| MDA-MB-468 | 0.800 | 28.798 | | 2.31E−06 |
| DU4475 | 1.028 | 24.079 | 1.20E−07 | 3.25E−06 |
| HCC1395 | 0.995 | 17.823 | 5.73E−09 | 8.14E−07 |
| EFM-19 | 0.000 | 9.103 | | 1.43E−05 |
| HCC1143 | 0.722 | 8.425 | | 4.60E−07 |
| HCC1187 | 1.101 | 8.379 | | 5.31E−07 |
| MCF7 F100-16 | 0.725 | 7.645 | | 1.73E−06 |
| MCF7 | 0.904 | 7.083 | | 1.12E−06 |
| HCC1937 | 0.805 | 6.868 | | 5.70E−07 |
| BT-20 | 0.521 | 6.323 | | 7.23E−07 |
| MDA-MB-157 | 1.174 | 6.213 | | 7.32E−07 |
| CAL-51 | 0.860 | 6.037 | | 1.36E−06 |
| MCF7 GHPED | 0.828 | 6.016 | | |
| HCC1569 | 0.778 | 5.909 | 2.21E−05 | 9.57E−07 |
| JIMT-1 | 0.792 | 5.637 | | 8.69E−07 |
| BT474C1 | 0.965 | 5.267 | | 3.83E−06 |
| MDA-MB-134IV | 0.906 | 4.430 | | 5.78E−06 |
| SUM52PE | 0.847 | 4.386 | | 6.51E−06 |
| HCC1806 | 0.917 | 3.698 | | 4.12E−07 |
| HCC1954 | 0.875 | 3.612 | | 2.56E−06 |
| SK-BR-3 | 1.070 | 2.298 | | 5.42E−07 |
| CAL-120 | 0.526 | 2.140 | | 1.18E−06 |
| MDA-MB-231 | 1.030 | 2.104 | | 8.66E−07 |
| T47D | | 1.730 | | 6.85E−06 |
| MCF7 T52 | 1.007 | 1.641 | 4.18E−08 | 1.39E−06 |
| ZR-75-1 | 0.869 | 1.293 | | 6.03E−06 |

### Lung Cell Lines (AZ14114554 & AZD6736)

| | | Synergy | AZ14114554 | AZD6738 |
|---|---|---|---|---|
| | Maximum | Score | mono | mono |
| Cell Line | CI | (HSA) | AC₅₀ (M) | AC₅₀ (M) |
| NCI-H2009 | 1.048 | 10.112 | 1.05E−07 | 1.02E−06 |
| NCI-H810 | 0.523 | 9.735 | | 3.35E−06 |
| NCI-H3122 | 0.836 | 6.993 | | 3.12E−06 |
| DMS53 | 0.712 | 6.842 | 6.22E−08 | 2.18E−06 |
| NCI-H1573 | 0.725 | 6.832 | 1.96E−08 | 1.39E−06 |
| PC-9 | 0.873 | 6.789 | 9.71E−05 | 5.10E−07 |
| NCI-H322 | 0.790 | 6.404 | 1.23E−09 | 1.56E−06 |
| NCI-H1993 | 1.047 | 6.282 | | 2.97E−06 |
| A549 | 0.997 | 4.414 | | 3.11E−06 |
| NCI-H441 | 0.988 | 4.322 | | 2.96E−06 |
| SW1271 | 1.071 | 3.453 | | 2.65E−06 |
| NCI-H1975 | 0.630 | 3.273 | | 5.28E−06 |
| NCI-H1395 | 1.069 | 2.459 | | 3.31E−06 |
| NCI-H1650 | 0.894 | 2.246 | 6.32E−08 | 1.05E−06 |
| Calu-1 | 1.006 | 2.170 | | 3.22E−06 |
| NCI-H2228 | | 1.907 | | 1.05E−05 |
| SW1573 | 0.967 | 1.705 | | 3.65E−06 |
| NCI-H2085 | 0.749 | 1.703 | | 4.75E−06 |
| NCI-H358 | 1.051 | 1.423 | | 2.67E−06 |
| NCI-H2122 | 0.815 | 1.351 | | 1.73E−06 |
| NCI-H2291 | 0.960 | 1.137 | | 1.12E−06 |

### Lung Cell Lines (AZ14114554 & AZD6736)

| | | Synergy | AZD5305 | AZD6738 |
|---|---|---|---|---|
| | Maximum | Score | mono | Mono |
| Cell Line | CI | (HSA) | AC₅₀ (M) | AC₅₀ (M) |
| NCI-H1573 | 0.788 | 7.966 | 4.77E−08 | 8.63E−07 |
| NCI-H23 | 0.675 | 6.803 | 4.13E−09 | 2.21E−07 |
| NCI-H522 | 0.637 | 6.629 | | 5.65E−06 |
| HCC15 | 0.221 | 6.545 | 7.70E−09 | 7.02E−06 |
| NCI-H1299 | 0.311 | 5.995 | | 3.18E−06 |
| NCI-H1650 | 0.711 | 5.286 | | 2.15E−06 |
| NCI-H1975 | 0.499 | 4.086 | | 1.89E−06 |
| NCI-H322 | 0.724 | 3.965 | | 6.54E−07 |
| NCI-H1373 | 0.418 | 3.948 | | 1.24E−06 |
| NCI-H3122 | 0.949 | 3.047 | | 3.21E−06 |
| SW1271 | 0.666 | 2.683 | | 6.69E−07 |
| NCI-H2122 | 0.990 | 2.590 | | 5.64E−07 |
| NCI-H2228 | | 2.570 | | 1.26E−06 |
| NCI-H1792 | 1.566 | 2.342 | 2.45E−08 | 6.77E−07 |
| NCI-H1395 | | 2.319 | | 2.90E−06 |
| Calu-1 | 0.632 | 2.290 | | 5.60E−06 |
| HOP62 | 0.507 | 2.029 | | 5.90E−07 |
| NCI-H838 | 1.842 | 1.495 | | 2.74E−07 |
| NCI-H2085 | 0.710 | 1.394 | | 5.49E−06 |
| LU99 | 1.026 | 1.380 | | 5.81E−07 |
| NCI-H1666 | | 1.038 | 9.36E−09 | 7.67E−07 |

### Example 2—In Vitro Combination Assay

Combination profiling was carried out in a panel of cancer cell lines utilising Horizon Discovery's High Throughput Screening platform. Growth inhibition was determined using a 144-hour CellTiter-Glo®2.0 proliferation assay.

Cell lines that have been preserved in liquid nitrogen are thawed and expanded in growth media (see Table 1). Once cells have reached expected doubling times, screening begins. Cells are seeded in 25 μl of growth media in black 384-well tissue culture treated plates (seeding density as noted in Table 11).

Cells are equilibrated in assay plates via centrifugation and placed at 37° C. 5% CO₂ for twenty-four hours before treatment. At the time of treatment, a set of assay plates (which do not receive treatment) are collected and ATP levels are measured by adding CellTiter-Glo 2.0 (Promega) and luminescence read on Envision plate readers (Perkin Elmer).

Compounds are transferred to assay plates using and Echo acoustic liquid handling system. 25 nl of each compound was added at the appropriate concentration for all combination dose points. Therefore, final assay volume would be 25.05 μl. Assay plates are incubated with compound for 6 days and are then analysed using CellTiter-Glo 2.0. All data points are collected via automated processes and are subject to quality control and analysed.

Growth Inhibition (GI) is reported as a measure of cell growth. The GI percentages are calculated by applying the following test and equation:

$$If T < V\_ 0 : 100 * ( 1 ( T - V\_ 0 ) / {V}_{-} 0 )$$

$$If T \geq V\_ 0 : 100 * ( 1 - ( T - V\_ 0 ) / ( V - V\_ 0 ) )$$

where T is the signal measure for a test article, V is the untreated/vehicle-treated control measure, and Vo is the untreated/vehicle control measure at time zero (also colloquially referred as T0 plates). This formula is derived from the Growth Inhibition calculation used in the National Cancer Institute's NCI-60 high throughput screen. For the purposes of this report, all data analysis was performed in Growth Inhibition (except where noted).

TABLE 1

| | | | Treatment | Seeding |
|---|---|---|---|---|
| | | | Time | Density |
| Cell Line | Tissue | Media | (h) | (cpw) |
| A2780 | Ovary | RPMI with 10% FBS | 29 | 250 |
| AGS | Gastric | Hams F12K with 10% FBS | 49 | 500 |
| BxPC-3 | Pancreas | RPMI with 10% FBS | 44 | 500 |
| Caov-3 | Ovary | DMEM with 10% FBS | 80 | 500 |
| CAPAN-2 | Pancreas | McCoy's 5A with 10% FBS | 68 | 500 |
| CFPAC-1 | Pancreas | IMDM with 10% FBS | 52 | 500 |
| COLO-201 | Colorectal | ATCC-formulated RPMI with 10% | 47 | 500 |
| | | FBS | | |
| COLO-205 | Colorectal | RPMI with 10% FBS | 34 | 500 |
| COLO-320- | Colorectal | RPMI with 10% FBS | 33 | 500 |
| HSR | | | | |
| COV362 | Ovary | DMEM with 10% FBS | 57 | 500 |
| DLD-1 | Colorectal | RPMI with 10% FBS | 36 | 500 |
| GCIY | Gastric | EMEM with 15% FBS | 54 | 500 |
| Gp2D | Colorectal | DMEM with 10% FBS | 43 | 500 |
| HCT-116 | Colorectal | McCoy's 5A with 10% FBS | 39 | 500 |
| HCT-15 | Colorectal | RPMI with 10% FBS | 34 | 500 |
| HGC-27 | Gastric | EMEM with 10% FBS | 35 | 500 |
| HPAF-II | Pancreas | EMEM with 10% FBS | 45 | 500 |
| HT-115 | Colorectal | DMEM with 15% FBS and 2 mM | 33 | 250 |
| | | Glutamine | | |
| IM-95 | Gastric | DMEM with 10% FBS and 10 mg/l | 52 | 500 |
| | | human Insulin | | |
| JHOM-1 | Ovary | DMEM:Ham's F12 (1:1) with 10% | 79 | 500 |
| | | FBS and 1% NEAA | | |
| KE-39 | Gastric | ATCC-formulated RPMI with 10% | 41 | 500 |
| | | FBS | | |
| KP-3 | Pancreas | RPMI with 10% FBS | 51 | 500 |
| KURAMOCHI | Ovary | RPMI with 10% FBS | 55 | 500 |
| LMSU | Gastric | Ham's F10 with 10% FBS | 46 | 500 |
| LoVo | Colorectal | Hams F12K with 10% FBS | 37 | 250 |
| LS-513 | Colorectal | RPMI with 10% FBS | 48 | 500 |
| MCAS | Ovary | EMEM with 20% FBS | 51 | 500 |
| MIA PaCa-2 | Pancreas | DMEM with 10% FBS and 2.5% | 48 | 500 |
| | | horse serum | | |
| MKN74 | Gastric | RPMI with 10% FBS | 50 | 500 |
| NCI-SNU-1 | Gastric | ATCC-formulated RPMI with 10% | 47 | 1500 |
| | | FBS | | |
| OV7 | Ovary | DMEM:Ham's F12 (1:1) with 5% | 59 | 500 |
| | | FBS, 0.5 ug/ml Hydrocortisone and | | |
| | | 10 ug/ml Human Insulin | | |
| OVCAR-3 | Ovary | RPMI with 20% FBS and 0.01 | 54 | 500 |
| | | mg/ml bovine insulin | | |
| OVK18 | Ovary | EMEM with 10% FBS | 46 | 500 |
| OVSAHO | Ovary | RPMI with 10% FBS | 46 | 500 |
| PA-1 | Ovary | EMEM with 10% FBS, 1% NEAA, | 41 | 500 |
| | | 1 mM Sodium Pyruvate and 1.5 g/l | | |
| | | sodium bicarbonate | | |
| PSN1 | Pancreas | RPMI with 10% FBS | 43 | 500 |
| RKO | Colorectal | EMEM with 10% FBS | 36 | 500 |
| SK-OV-3 | Ovary | McCoy's 5A with 10% FBS | 45 | 500 |
| SNU-324 | Pancreas | ATCC-formulated RPMI with 10% | 60 | 500 |
| | | FBS, 25 mM HEPES and 25 mM | | |
| | | Sodium | | |
| | | Bicarbonate | | |
| SNU-668 | Gastric | ATCC-formulated RPMI with 10% | 43 | 500 |
| | | FBS, 25 mM HEPES and 25 mM | | |
| | | Sodium | | |
| | | Bicarbonate | | |
| SW620 | Colorectal | RPMI with 10% FBS | 38 | 500 |
| SW837 | Colorectal | RPMI with 10% FBS | 44 | 500 |
| TOV-21G | Ovary | MCDB 105:MEDIUM 199 (1:1) | 55 | 500 |
| | | with 15% FBS and 1.5 G/L Sodium | | |
| | | Bicarbonate | | |

### Pancreatic Cell Lines (AZD9574 & AZD6738)

| | | | AZD6738 |
|---|---|---|---|
| | | Synergy Score - | mono GI₅₀ |
| Cell Line | Max CI | HSA | (μM) |
| KP-3 | 1.64 | 2.48 | 1.2828 |
| MIA PaCa-2 | 1.39 | 4.05 | 1.2037 |
| PSN1 | 1.92 | 1.35 | 0.8585 |
| CFPAC-1 | 1.45 | 3.86 | 0.7569 |
| SNU-324 | | 2.5 | 0.4761 |
| BxPC-3 | 1.51 | 2.36 | 1.0844 |

### Pancreatic Cell Lines (AZD5305 & AZD6738)

| | | | AZD6738 |
|---|---|---|---|
| | | Synergy Score - | mono GI50 |
| Cell Line | Max CI | HSA | (μM) |
| MIA PaCa-2 | 1.3 | 6.65 | 1.9294 |
| PSN1 | 1.25 | 4.02 | 0.7455 |
| KP-3 | 1.61 | 4.46 | 1.8987 |
| SNU-324 | 1.94 | 3.11 | 0.5336 |
| BxPC-3 | 1.38 | 1.87 | 1.0883 |
| HPAF-II | 1.41 | 1.9 | 1.0262 |
| CAPAN-2 | 1.68 | 2.28 | |

### Ovarian Cell Lines (AZD9574 & AZD6738)

| | | | AZD6738 |
|---|---|---|---|
| | | HSA | mono GI50 |
| Cell Line | Max CI | volume | (μM) |
| OVCAR-3 | 1.17 | 10.92 | 0.3703 |
| PA-1 | 1.51 | 9.09 | 1.2243 |
| COV362 | 1.16 | 2.38 | 1.0553 |
| KURAMOCHI | 1.27 | 5.78 | 0.7285 |
| OVK18 | 1.38 | 4.42 | 0.9209 |
| A2780 | 1.33 | 5.81 | 0.6667 |
| TOV-21G | 1.48 | 3.13 | 0.3741 |
| JHOM-1 | 1.55 | 3.83 | 0.9387 |
| OVSAHO | 1.7 | 4.33 | 0.5806 |
| SK-OV-3 | 1.37 | 3.15 | 1.2771 |
| OV7 | 1.73 | 2.06 | 1.9247 |
| MCAS | | 1.78 | |

### Ovarian Cell Lines (AZD5305 & AZD6738)

| | | | | AZD6738 |
|---|---|---|---|---|
| | Best CI | | HSA | mono GI50 |
| Cell Line | Level | Max CI | volume | (μM) |
| OVCAR-3 | 150 | 1.26 | 11.79 | 0.3703 |
| Caov-3 | 95 | 1.27 | 10.75 | 0.1968 |
| PA-1 | 125 | 1.35 | 8.47 | 1.2243 |
| COV362 | 85 | 1.1 | 12.85 | 1.0553 |
| OVK18 | 50 | 1.29 | 7.92 | 0.9209 |
| KURAMOCHI | 75 | 1.22 | 8.34 | 0.7285 |
| JHOM-1 | 85 | 1.48 | 3.91 | 0.9387 |
| A2780 | 70 | 1.26 | 6.01 | 0.6667 |
| TOV-21G | 55 | 1.27 | 5.22 | 0.3741 |
| SK-OV-3 | 60 | 1.66 | 4.49 | 1.2771 |
| OVSAHO | 25 | | 2.39 | 0.5806 |
| OV7 | 50 | 1.7 | 1.06 | 1.9247 |

### Gastric Cell Lines (AZD9574 & AZD6738)

| | | | AZD6738 |
|---|---|---|---|
| | | HSA | mono GI50 |
| Cell Line | Max CI | volume | (μM) |
| IM-95 | 1.34 | 2.39 | 0.2501 |
| GCIY | 1.64 | 7.09 | 0.1786 |
| LMSU | 1.09 | 5.26 | 1.7108 |
| SNU-668 | 1.27 | 2.5 | 0.2907 |
| MKN74 | 1.26 | 4.16 | 0.7406 |
| NCI-SNU-1 | 1.27 | 4.42 | 1.0924 |
| KE-39 | 1.26 | 4.11 | 1.4747 |
| AGS | 1.33 | 4.34 | 1.2065 |

### Gastric Cell Lines (AZD5305 & AZD6738)

| | | | AZD6738 |
|---|---|---|---|
| | | HSA | mono GI₅₀ |
| Cell Line | Max CI | volume | (μM) |
| IM-95 | 1.25 | 10.24 | 0.2501 |
| HGC-27 | 1.36 | 8.73 | 1.1505 |
| LMSU | 1.07 | 13.82 | 1.7108 |
| MKN74 | 1.29 | 8.49 | 0.7406 |
| SNU-668 | 1.28 | 7.68 | 0.2907 |
| GCIY | 1.44 | 6.42 | 0.1786 |
| NCI-SNU-1 | 1.25 | 5.69 | 1.0924 |
| KE-39 | 1.39 | 6.53 | 1.4747 |

### Colorectal Cell Lines (AZD9574 & AZD6738)

| | | | AZD6738 |
|---|---|---|---|
| | | HSA | mono GI₅₀ |
| Cell Line | Max CI | volume | (μM) |
| HCT-116 | 1.19 | 4.71 | 0.6871 |
| HT-115 | 1.21 | 2.45 | 1.6057 |
| Gp2D | 1.44 | 3.36 | 0.5008 |
| HCT-15 | 1.31 | 1.89 | |
| LS-513 | 1.45 | 2.97 | 0.6453 |
| SW620 | 1.26 | 5.44 | 0.8531 |
| COLO-205 | 1.62 | 1.12 | |
| SW837 | 1.61 | 5.68 | 0.9238 |
| LoVo | 2 | 1.66 | 0.4009 |
| RKO | 1.92 | 3.21 | 1.0177 |
| COLO-201 | | 1.07 | |

### Colorectal Cell Lines (AZD5305 & AZD6738)

| | | | AZD6738 |
|---|---|---|---|
| | | HSA | mono GI₅₀ |
| Cell Line | Max CI | volume | (μM) |
| HCT-116 | 1.1 | 15.33 | 0.6871 |
| COLO-320-HSR | 1.1 | 10.48 | |
| HT-115 | 1.15 | 7.75 | 1.6057 |
| Gp2D | 1.35 | 7.21 | 0.5008 |
| SW620 | 1.34 | 3.61 | 0.8531 |
| HCT-15 | 1.11 | 4.79 | |
| COLO-205 | 1.55 | 1.55 | |
| LoVo | 1.64 | 2.9 | 0.4009 |
| DLD-1 | 1.34 | 2.94 | |

### Example 3—In Vitro Combination Assay

The assay was carried out in the following glioblastoma cell lines:
U87
U87 R132H
T98G
SJ-G2 ctrl
SJ-G2 IDH
   using AZD6738 and AZD9574

### Treatment: 7 Days Treatment

### Method

The cells were seeded the day before drug treatment in 150 μl per well of 96-well plate. For SJ-G2 cells, the plates were coated with poly-Lysine solution for 15 minutes, washed twice with sterile water and dried for 1 hour.

The seeding numbers were (cells per well): U87-500; T98G-500; SJ-G2 ctrl-1000; SJ-G2 IDH.

The compounds were added via a drug dispenser according to the following scheme:

Plate 1

| | | | | | | | | |
|---|---|---|---|---|---|---|---|---|
| 2 | 3 | 4 | 5 | 6 | 7 | 8 | 9 | 10 |

| | | | |
|---|---|---|---|
| B | DMSO | 10 nM C2 | 30 nM C2 |
| C | 30 nM C1 | 30 nM C1; 10 nM C2 | 30 nM C1; 30 nM C2 |
| D | 100 nM C1 | 100 nM C1; 10 nM C2 | 100 nM C1; 30 nM C2 |
| E | 300 nM C1 | 300 nM C1; 10 nM C2 | 300 nM C1; 30 nM C2 |
| F | 534 nM C1 | 534 nM C1; 10 nM C2 | 534 nM C1; 30 nM C2 |
| G | 1000 nM C1 | 1000 nM C1; 10 nM C2 | 1000 nM C1; 30 nM C2 |

Plate 2

| | | | | | | | | |
|---|---|---|---|---|---|---|---|---|
| 2 | 3 | 4 | 5 | 6 | 7 | 8 | 9 | 10 |

| | | | |
|---|---|---|---|
| B | 100 nM C2 | 300 nM C2 | 1000 nM C2 |
| C | 30 nM C1; 100 nM C2 | 30 nM C1; 300 nM C2 | 30 nM C1; 1000 nM C2 |
| D | 100 nM C1; 100 nM C2 | 100 nM C1; 300 nM C2 | 100 nM C1; 1000 nM C2 |
| E | 300 nM C1; 100 nM C2 | 300 nM C1; 300 nM C2 | 300 nM C1; 1000 nM C2 |
| F | 534 nM C1; 100 nM C2 | 534 nM C1; 300 nM C2 | 534 nM C1; 1000 nM C2 |
| G | 1000 nM C1; 100 nM C2 | 1000 nM C1; 300 nM C2 | 1000 nM C1; 1000 nM C2 |

Compound C1 - AZD6738;
Compound C2 - AZD9574

after 7 Days:
(a) for SJ-G2, 75 μl Cell Titer Glow 2.0 solution (Promega) was added and incubated for 15 minutes at 37° C. The cells were then read using a plate reader.
(b) for U87 and T98G, 75 μl of 37% formaldehyde was added and fixed for 20 minutes at room temperature. This was then washed twice with PBS, followed by Hoeschst staining for 1 hour at room temperature (Hoeschst 1:10000 in PBS). This was then washed twice in PBS and the plates sealed. The plates were then imaged on Cell Insight with 10× magnification at the 25 position. Hoeschst positive nuclei were identified as objects, and cell survival was analysed by counting objects (Hoeschst positive nuclei) with the corresponding settings for U87 or T98G.

An analysis of drug synergy was then carried out to yield a HSA score

### Results

| | | | AZD67385 |
|---|---|---|---|
| Cell line | Details | HSA score | IC₅₀ (μM) |
| U87 IDH WT | Adult GBM | 5.022 | 0.649 |
| U87 IDHm | Adult GBM | 13.755 | 0.766 |
| SJ-G2 WT | paediatric GBM | 37.750 | 0.16 |
| SJ-G2 IDHm | paediatric GBM | 20.760 | 0.308 |
| T98G | Adult GBM | 45.528 | 0.213 |

### Example 4—In Vitro Combination in Isogenic PARP Inhibitor Resistant Cell Line Pairs

Many of the described BRCA/HRR-dependent mechanisms of acquired PARP inhibitor resistance centre on the restoration of HRR by either reversion mutations or HR rewiring such as through alterations on other DDR components e.g. loss of 53BP1/Shieldin complex (Prados Carvajal 2021). In a BRCA1 mutant breast cancer cell line the knockout of the TP53BP1 gene (53BP1 protein) by CRISPR-Cas9 technology conferred resistance to PARP-inhibitor monotherapy but remained sensitive to the combination of AZD5305 with ceralasertib, overcoming this resistance mechanism.

### Method

### CRISPR-Cas9 TP53BP1 WT and Knockout (KO) Cell Line Generation

Parental BRCA1 mutant SUM149PT breast cancer cell line (Elstrodt 2006) were obtained from AstraZeneca Cell Bank at Alderley Park, Macclesfield. Cells were routinely grown in Ham's F12 media containing 5% foetal calf serum, 1% glutamine (2 mM), 500 ng/ml hydrocortisone and 5% insulin. SUM149PT 53BP1 WT (CNTR) and 53BP1 null (KO) cell pools were generated by CRISPR-Cas9 technology. Short guide (sg) RNA targeting TP53BP1 on exon 10 (GAGTAGATCGGAAAGCATC) and a non-targeting CNTR guide (GAGTAGATCGGAAAGCATC) were designed and cloned into a lentiviral vector containing a mClover3 cassette for green fluorescence signal and hygromycin cassette for selection (pKLV2-U6gRNA (Bbsl)-EF1a-mClover3-T2A-HygR-W). Lentiviruses were generated from both KO and CNTR (sg) RNA and Cas9 (pKLVEF1a-Cas9Bsd-W) plasmids and parental cells were transduced first with Cas9 lentiviruses followed by blasticidin selection and then with KO and CNTR lentiviruses followed by hygromycin selection. Loss of 53BP1 was verified by analysing 53BP1 protein expression from whole cell lysates by western blotting (Novus, NB100-304, 1:1000 dilution).

### Clonogenic Proliferation Assays for Monotherapy and Combination Treatments

Cells were seeded onto 6-well plates, three replicates per cell line, and 24 hours later the cells were dosed with AZD6738/AZD5305. For monotherapies the cells were dosed with a 6-point concentration response of AZD6738 (0 to 0.64 μM) or AZD5305 (0 to 1 μM). For combination treatments single dose of AZD5305 (10 nM) was used with a 5-point concentration response of AZD6738 (0 to 0.64 μM) for 53BP1 KO cells. Cells were grown for 14 days for colony formation without media changes. Cells were fixed with 10% TCA (Tricloroacetic acid). 14 days after dosing any formed colonies were stained with 0.057% SRB (sulforhodamine B acid), imaged with GelCount™ (Oxford OPTRONIX) at 600 dpi resolution and the stained colony growth intensity was measured using plate reader with OD @510 nM. Dose response curves were generated and IC₅₀ concentrations were calculated using GraphPad PRISM software.

### Results

CRISPR-Cas9 technology was used to knockout (KO) TP53BP1 in SUM149PT cell line, a control (CNTR) cell line was generated in parallel using a non-targeting guide.

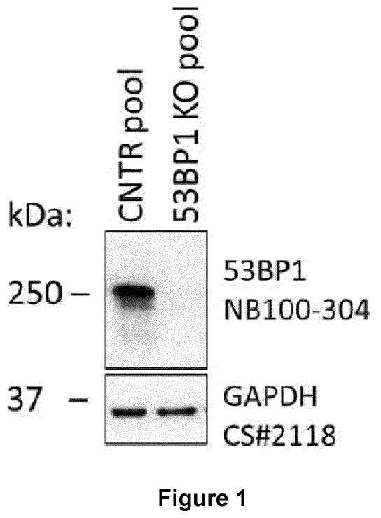

FIG. 1 confirms that the SUM149PT 53BP1 KO cell pool is null for 53BP1 protein expression when compared to the CNTR cell pool. GAPDH protein expression shows that the protein lysate loading was equal for both cell pools.

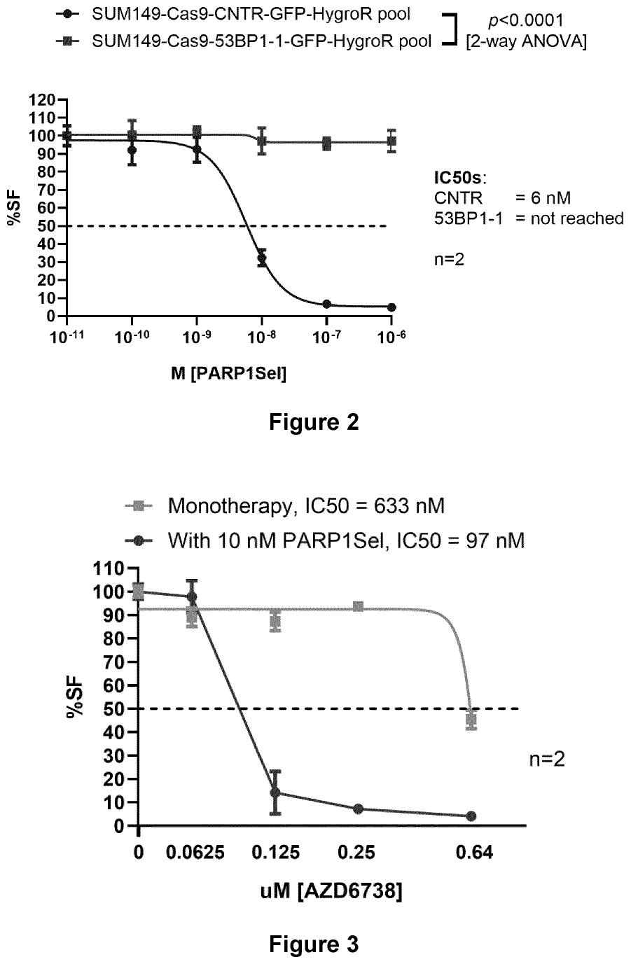

FIG. 2 shows clonogenic growth assays confirming that the loss of 53BP1 in SUM149PT cells causes a significant increase in resistance to AZD5305. SUM149PT 53BP1 WT (CNTR) cells are highly sensitive to AZD5305 (IC₅₀ ˜6 nM). In contrast, the SUM149PT 53BP1-1 KO cell pools become completely resistant to AZD5305.

FIG. 3 shows clonogenic growth assays where the PARP inhibitor resistant SUM149PT 53BP1 KO cell pools are sensitive to the combination of AZD6738 with AZD5305. AZD6738 monotherapy shows modest activity with IC₅₀ of ˜0.63 UM in both CNTR and 53BP1 KO cell pools (53BP1 does not affect AZD6738 monotherapy sensitivity). However, the combination of a single fixed low dose of 10 nM AZD5305 (a dose which does not show growth inhibition as monotherapy) with a AZD6738 dose response shows strong, synergistic enhancement of growth inhibition with a ˜6-fold decrease in IC₅₀ in CNTR (IC₅₀ ˜0.11 μM) and 53BP1 KO (IC₅₀ ˜0.097 μM) cell pools.

The aim of the study is to determine the Maximum Tolerated Dose (MTD), which will be determined as the highest dose at which the predicted probability of a Dose-limiting toxicities (DLT) is 30% (±5%) during the DLT review period.

A DLT is defined as any toxicity during the periods of Cycle 0 and Cycle 1 (i.e. from dosing on Cycle 0 Day 1 until the last day of dosing in Cycle 1), which includes:
1. Haematological toxicities as follows:
   Grade 4 neutropenia (ANC (Absolute neutrophil count)<500 cells/mm³) lasting longer than 4 consecutive days
   Grade 3 neutropenia (ANC≥500 to <1000 cells/mm³) of any duration accompanied by fever ≥38.5° C. and/or systemic infection
   Grade 3 thrombocytopenia (25,000 to <50,000/mm³) with bleeding
   Any other confirmed haematological toxicity≥CTCAE (Common Terminology Criteria for Adverse Events) version 5 Grade 4 (a repeat may be required for confirmation of an isolated abnormality in the absence of clinical signs, symptoms or other abnormal investigations, ie, a suspected spurious value)
2. Non-haematological toxicity≥ CTCAE version 5 Grade 3 including:
   Laboratory abnormalities (a repeat may be required for confirmation of an isolated abnormality in the absence of clinical signs, symptoms or other abnormal investigations, ie, a suspected spurious value)
   Nausea, vomiting or diarrhoea lasting for >72 hours despite administration of maximal supportive therapy
   Cardiac DLTs including:
      Symptomatic tachycardia or tachycardia with resting supine heart rate of >125 beats per minute persisting for at least 10 minutes
      Hypotension requiring medical intervention eg, IV fluids
      Any other cardiac toxicity of CTCAE>Grade 2.
      QTcF (QT interval corrected for heart rate using Fridericia's formula) interval value<340 msec confirmed on at least 2 separate ECGs (Electrocardiograms), recorded 5 minutes apart
      QTcF prolongation>500 msec or QTcF prolongation from baseline by >60 msec confirmed on at least 2 separate ECGs, recorded 5 minutes apart
3. Any other toxicity that is greater than that at baseline, and is clinically significant and/or unacceptable, and does not respond to supportive care
4. Any event, including significant dose reductions or omissions, judged to be a DLT. Examples may include confirmed laboratory abnormalities (CTCAE Grade≥3), CTCAE Grade 2 toxicities that are clinically significant and/or unacceptable according to the investigator, toxicities that result in an inability to administer at least 75% of study treatment during Cycle 1 or delay the administration of study treatment in the subsequent cycle by ≥7 consecutive days.
   Any death not clearly due to the underlying disease or extraneous causes.
      A DLT excludes:
Alopecia of any grade.

For part B, a first expansion cohort will be initiated once dose level 2 escalation (160 mg BID 14 days combined with continuous AZD5305 2.5 mg QD) is tolerated, whilst continuing in parallel with further dose escalations. Once the dose of 160 mg BD 14 days of ceralasertib combined with continuous AZD5305 5 mg (dose level 3) is tolerated, a second expansion cohort will be triggered, and the first expansion cohort at dose level 2 may be stopped.

At the end of the study, the recommended Phase 2 doses will be determined.

AZD5305 and ceralasertib are taken as separate tablets, on an empty stomach, with no food two hours before, and for at least one hour afterwards. Ceralasertib will be dosed using film-coated tablets containing either 20 or 80 mg of ceralasertib. AZD5305 will be dosed using film-coated tablets containing either 0.5 or 5 mg of AZD5305.

### Ceralasertib and AZD5305 PK Blood Sampling Schedule for Part A

| Time relative to dose^{a} | C0D 1^{b} | C1D 1 | C1D 8 | C2D 15 | IP disc. |
|---|---|---|---|---|---|
| Pre-dose (−30 min ± 15 min) | X | X | X | X^{c} | |
| 0.5 h (±5 min) | X | X | X | | |
| 1 h (±15 min) | X | X | X | | |
| 1.5 h (±15 min) | X | X | X | | |
| 2 h (±15 min) | X | X | X | | |
| 3 h (±30 min) | X | X | X | | |
| 4 h (±30 min) | X | X | X | | |
| 8 h (±60 min) | X | X | X | | |
| 10 h (±60 min) | X | X | X | | |
| 24 h (±60 min)^{d} | X | X | X | | |
| 0-96 h | | | | | X^{e} |

^{a}Times of samples relative to dose are measured relative to the morning dose on C0D 1, C1D 1, C1D 8, and C2D 15.
^{b}Only for ceralasertib.
^{c}No ceralasertib dose on D 15. Sample should be taken 30 min prior to AZD5305 dose.
^{d}The 24 h sample on D 1 should be collected prior to the D2 dose where relevant.
^{e}A discontinuation sample is to be collected wherever possible between 0 and 96 hours post last dose.
C = cycle; D = day; disc. = discontinuation; h = hour(s); IP = investigational product; min = minutes; PK = pharmacokinetic.

### Ceralasertib and AZD5305 PK Blood Sampling Schedule for Part B

| Time relative to dose^{a} | C1D 1 | C1D 15 | C2D 15 | IP disc. |
|---|---|---|---|---|
| Pre-dose (−30 min ± 15 min) | X | X^{b} | X | |
| 0.5 h (±5 min) | X | X^{c} | | |
| 1 h (±15 min) | X | X^{c} | | |
| 2 h (±15 min) | X | X^{c} | | |
| 4 h (±30 min) | X | X^{c} | | |
| 6 h (±30 min) | X | X^{c} | | |
| 0-96 h | | | | X^{e} |

^{a}Times of samples relative to dose are measured relative to the morning dose on C1D 1, C1D 15, and C2D 15.
^{b}No ceralasertib dose on D 15. Sample should be taken 30 min prior to AZD5305 dose.
^{c}Only for AZD5305.
^{d}The 24 h sample on D 1 should be collected prior to the D2 dose where relevant.
^{e}A discontinuation sample is to be collected wherever possible between 0 and 96 hours post last dose.

Where possible, the following PK parameters will be determined for ceralasertib and AZD5305 at the time points outlined above.

Following the single dose:
AUC₍₀₋ₜ₎ [Area under the plasma concentration-time curve from zero to the last measurable time point]
AUC₍₀₋₆₎ (Part B only) [Area under the plasma concentration-time curve from zero to 6 hours]
AUC₍₀₋₂₄₎ [Area under the plasma concentration-time curve from zero to 24 hours]
AUCᵢₙₜ [Area under the plasma concentration-time curve from zero to infinity]
AUC_{T} [Area under the plasma concentration-time curve from zero to the end of the dosing
Interval]
Cₘₐₓ [Maximum plasma concentration]
tₘₐₓ [Time to maximum plasma concentration]
CL/F [Apparent plasma clearance]
λ_{z} [Terminal phase elimination rate constant]
t_{1/2,λz} [Drug elimination during the terminal phase]
V₂/F [Volume of distribution]

Following multiple dosing:
C_{ss max} [Maximum plasma concentration at steady state]
t_{ss max} [Time to maximum plasma concentration at steady state]
C_{ss min} [Minimum plasma concentration at steady state]
AUCₛₛ [Area under the plasma concentration-time curve from zero to the end of the dosing
Interval]
AUC₍₀₋ₜ₎
AUC₍₀₋₆₎ (Part B only)
AUC₍₀₋₂₄₎
AUC_{inf}
AUC_{T}
CLₛₛ/F [Apparent plasma clearance at steady state]
R_{AC} [Extent of accumulation on multiple dosing]
λ_{z}
t_{1/2,λz}
Vₛₛ/F [Volume of distribution]
Time dependency of the PK

The Cₘₐₓ, the C_{ss max}, the tₘₐₓ and the t_{ss max} will be determined by inspection of the concentration-time profiles. Where possible, the Az will be calculated by log-linear regression of the terminal portion of the concentration-time profiles where there are sufficient data and the t_{1/2λz} will be calculated as ln 2/λ_{z}. All AUC-related parameters following single and multiple dosing will be calculated using the linear up/log down trapezoidal rule. Where appropriate, the AUC will be extrapolated to infinity using λ_{z} to obtain AUC_{inf}. The CL/F following the single dose and CLₛₛ/F following multiple dosing will be determined from the ratio of dose/AUC or dose/AUCₛₛ. The Vₛₛ/F or V_{z}/F will be determined from the MRT×CL/F and/or the R_{AC} will be calculated as the ratio of the AUC₍₀₋₂₄₎ and/or Cₘₐₓ on Cycle 1 Day 8 and Cycle 1 Day 1. The time dependency of the PK on multiple dosing will be assessed by the calculation of the ratio of AUC, Cycle 1 Day 8 and AUC_{inf} on Cycle 1 Day 1.

### Anti-Tumour Activity

Baseline tumour assessments should encompass all areas of known predilection for metastases in the disease under evaluation and should additionally investigate areas that may be involved based on signs and symptoms of individual participants. Baseline assessments should be performed no more than 28 days before the start of study treatment, and ideally should be performed as close as possible to the start of study treatment. The methods of assessment used at baseline should be used at each subsequent follow-up assessment. Follow-up assessments should be performed every 8 weeks (+1 week) after the start of combination treatment (Cycle 1 Day 1) until objective disease progression as defined by RECIST version 1.1 (Eisenhauer 2009) or withdrawal of consent. Once a participant has received ceralasertib for over 2 years and their tumour is not changing in size (SD, PR or CR), the frequency of their RECIST version 1.1 assessment may be revised to every 16 weeks (±1 week) as judged by the investigator at local site based on an overall assessment of the benefit/risk, e.g. exposure to radiation. This decision should be documented in the participant's medical record.

Categorisation of objective tumour response assessment will be based on the RECIST version 1.1 guidelines for response: CR (complete response), PR (partial response), SD (stable disease) and PD (progressive disease).

To achieve ‘unequivocal progression’ on the basis of non-target disease, there must be an overall level of substantial worsening in non-target disease such that, even in presence of SD or PR in target disease, the overall tumour burden has increased sufficiently to merit discontinuation of therapy. A modest ‘increase’ in the size of one or more NTL is usually not sufficient to qualify for unequivocal disease progression status.

### Calculation or Derivation of Tumour Response Variables

At each tumour assessment visit participants will be programmatically assigned a RECIST version 1.1 visit response of CR, PR, SD or PD depending on the status of their disease compared with baseline and previous visit assessments.

Progression of TLs (target lesions) will be calculated in comparison to when the tumour burden was at a minimum (i.e. smallest sum of diameters previously recorded on study, including baseline). In the absence of progression, tumour response (CR, PR, SD) will be calculated in comparison to the baseline tumour measurements obtained before starting treatment.

If a participant has had a tumour assessment which cannot be evaluated, then the participant will be assigned a visit response of NE unless there is evidence of progression in which case the response will be assigned as PD.

For TL measurements, if ≤⅓ of the TL sizes are missing then a scaling up rule will be applied as follows:
If ≤⅓ of lesions recorded at baseline are missing then the results will be scaled up (based on the nadir sizes, including baseline) to give an estimated sum of diameters and this will be used in calculations (this is equivalent to comparing the visit sum of diameters of the non-missing lesions to the nadir sum of diameters excluding the lesions that are missing and determining at what rate the lesions are changing).
If >⅓ of lesions recorded at baseline are missing, then the TL response will be NE.
However, if the sum of non-missing TL diameters would result in PD (i.e. if using a value of 0 for missing lesions the sum of diameters has still increased by >20% or more compared to the smallest sum of diameters on study), PD takes precedence over NE.

A visit response of CR is defined when all TL and NTL lesions present at baseline have disappeared (with the exception of lymph nodes which must be <10 mm to be considered non-pathological) and no new lesions have developed since baseline. A visit response of PR is defined when the sum of diameters of the TLs has decreased by 30% or more compared to baseline (with no evidence of progression) and the NTLs are at least stable with no evidence of new lesions. To be assigned a status of PR or CR, changes in tumour measurements must be confirmed by repeat assessments that should be performed no less than 4 weeks after the criteria for response are first met.

Stable disease is defined as neither sufficient shrinkage to qualify for PR nor sufficient increase to qualify for PD. In the case of SD, follow-up measurements must have met the SD criteria at least once after study entry at a minimum interval of not less than 35 days.

### Objective Response Rate

Objective response rate is defined as the percentage of participants who have at least one response of CR or PR prior to any evidence of progression (as defined by RECIST version 1.1) that is confirmed at least 4 weeks later. For the analysis of ORR (overall response rate) an ‘evaluable for response’ population will be derived and will exclude participants who do not have measurable disease at baseline.

### Duration of Response

Duration of response will be defined as the time from the date of first documented response until date of documented progression or death in the absence of disease progression, the end of response should coincide with the date of progression or death from any cause used for the PFS endpoint. The time of the initial response will be defined as the latest of the dates contributing towards the first visit response of PR or CR.

If a participant does not progress following a response, then their DoR will use the PFS censoring time.

### Progression Free Survival

Progression free survival is defined as the time from start of treatment (first dose of ceralasertib) until the date of objective disease progression or death (by any cause in the absence of progression) regardless of whether the participant withdraws from therapy or receives another anti-cancer therapy prior to progression. Participants who have not progressed or died at the time of analysis will be censored at the time of the latest date of assessment from their last evaluable RECIST version 1.1 assessment. However, if the participant progresses or dies after two or more missed visits, the participant will be censored at the time of the latest evaluable RECIST version 1.1 assessment. If the participant has no evaluable visits or does not have baseline data, they will be censored at 0 days unless they die within 2 visits of baseline.

The PFS time will always be derived based on scan/assessment dates, not visit dates. RECIST version 1.1 assessments/scans contributing towards a particular visit may be performed on different dates. The following rules will be applied:
Date of progression will be determined based on the earliest of the dates of the component that triggered the progression.
When censoring a participant for PFS the participant will be censored at the latest of the dates contributing to a particular overall visit assessment.

Survival status will be obtained from all participants who received ceralasertib and AZD5305 until the data cutoff for the final analysis is reached. Survival status will be collected every 12 weeks (±1 week) for all participants. To aid the interpretation of the survival analysis, the use of subsequent anti-cancer therapies after discontinuation of study treatment will also be recorded on the eCRF (Electronic case report form) for participants who received ceralasertib and AZD5305. Survival status will continue to be collected until the earlier of 24 months after the last participant is recruited to Part B or when 80% of participants in each of the Part B cohorts are deceased.

### Overall Survival

Overall survival is defined as the time from the date of Cycle 0 Day 1 until death due to any cause. Any participant not known to have died at the time of analysis will be censored based on the last recorded date on which the participant was known to be alive.

### Inclusion Criteria

1. Histologically/cytologically confirmed, high grade epithelial ovarian, fallopian tube, or primary peritoneal cancer. Eligible histologies include high grade serous and high grade endometrioid. Ineligible histologies include low grade serous, low grade endometrioid, mucinous and carcinosarcoma.
2. Platinum-sensitive relapsed ovarian cancer:
   Platinum-sensitive disease is defined as no clinical or radiographic evidence of disease progression for >6 months (or 182 days) after last receipt of platinum-based therapy. The date should be calculated from the last administered dose of platinum therapy.
3. Participants must have been treated with prior PARPi.
a) Dose escalation (Part A): No requirement for response on prior PARPi but minimum treatment duration of 8 months (first line) or 4 months (second line) on prior PARPi.
b) Dose expansion (Part B): Either CR or PR on PARPi treatment, or minimum PARPi treatment duration of 8 months (first line) or 4 months (second line).
4. Mandatory washout period for any PARPi which should be 14 days or 5 half-lives (whichever is longer).
5. Progressive cancer at the time of study entry.
6. At least one lesion, preferably not previously irradiated, that can be accurately measured at baseline as ≥10 mm in the longest diameter (except lymph nodes which must have short axis≥15 mm) with CT or MRI and which is suitable for accurate repeated assessment. Previously irradiated lesions may be considered if there has been disease progression since radiotherapy.
7. Dose expansion only (Part B): Documentation of a known or suspected pathogenic BRCA mutation (either germline or somatic), or PALB2 mutation or RAD51C/D mutation (either germline or somatic) on a CAP/CLIA or other jurisdiction-appropriate certified assay according to local testing or HRD-positive status (Myriad MyChoice HRD+ assay using GIS at or greater 42 or FMI F1CDx assay using gLOH≥16). Submission of a copy of test result is mandatory for eligibility and enrolment. Prospective central HRD testing may be offered to sites depending on local testing availability.
8. Female participants must be using adequate contraceptive measures must not be breast feeding and must have a negative pregnancy test prior to start of dosing if of child-bearing potential or must have evidence of non-child-bearing potential by fulfilling one of the following criteria at screening:
a) Post-menopausal, defined as aged more than 50 years and amenorrhoeic for at least 12 months following cessation of all exogenous hormonal treatments
b) Documentation of irreversible surgical sterilisation by hysterectomy, bilateraloophorectomy or bilateral salpingectomy but not tubal ligation.
c) Amenorrhoeic for 12 months and serum FSH, LH and plasma oestradiol levels in the postmenopausal range for the institution.
9. Participants receiving NOACs (International normalised ratio) may be enrolled with an INR (International normalised ratio)<2; participants with other clinical causes of INR increase (such as bleeding disorders, impaired hepatic synthesis) should be excluded.

### REFERENCES

A number of publications are cited above in order to more fully describe and disclose the invention and the state of the art to which the invention pertains. Full citations for these references are provided below. The entirety of each of these references is incorporated herein.

| | Reference | Doi |
|---|---|---|
| Balasubramaniam | Balasubramaniam, S.; et al., Clin. | 10.1158/1078-0432.CCR-17-1337 |
| 2017 | Cancer Res. 2017, 23, 7165-7170 | |
| Barnieh 2021 | Barnieh F, et al., Curr Res Pharma | 10.1016/j.crphar.2021.100017 |
| | Drug Discov, 2021, 100017 | |
| Burgess 2020 | Burgess B, et al., Diagnostics 2020, | 10.3390/diagnostics10020121 |
| | 10, 121 | |
| Chappuis and | Chappuis P O and Foulkes W D, | 10.1007/978-1-4757-3587-1_2 |
| Foulkes 2002 | Cancer Treat Res, 107, 29-59 (2002) | |
| Charrier 2011 | Charrier J-D et al., J Med Chem, | 10.1021/jm101488z |
| | 2011, 54, 2320-2330 | |
| Cimprich 2007 | Cimprich K A, and Cortez D, Nat Rev | 10.1038/nrm2450 |
| | Mol Cell Biol 2008; 9: 616-27 | |
| Eisenhauer 2009 | Eisenhauer, E. A., et al., Eur Journal | 10.1016/j.ejca.2008.10.026 |
| | of Cancer (2009), 45, 228-247 | |
| Elstrodt 2006 | Elstrodt F, et al., Cancer Res, 2006; | 10.1158/0008-5472.CAN-05-2853 |
| | 66: 41-5 | |
| Foote 2013 | Foote K, et al., J. Med. Chem. (2013) | 10.1021/jm301859s |
| | 56, 2125-2138 | |
| Foote 2015 | Foote K M, et al., Future Med Chem | 10.4155/fmc.15.33 |
| | 2015; 7: 873-891. | |
| Foote 2018 | Foote K M, et al., J. Med. Chem. | 10.1021/acs.jmedchem.8b01187 |
| | 2018, 61, 9889-9907 | |
| Forment 2018 | Forment J V, and O'Connor M J, | 10.1016/j.pharmthera.2018.03.005 |
| | Pharmacol Ther 2018; 188: 155-167 | |
| George 2018 | George, E., et al., Gynecologic | |
| | Oncology 2018; 149: 45. SGO 2018 | |
| | conference (Poster 86) | |
| Gilad 2020 | Gilad, O., et al., 2020 Journal of | 10.1200/JCO.2020.38.15_suppl.e18000 |
| | Clinical Oncology 38, no. 15_suppl | |
| | 318000 | |
| Gorecki 2020 | Gorecki L et al., Pharmacology and | 10.1016/j.pharmthera.2020.107518 |
| | Therapeutics, 210 (2020), 107518 | |
| Hughes-Davies | Hughes-Davies, et al., Cell, 115, | 10.1016/s0092-8674(03)00930-9 |
| 2003 | 523-535 (2003) | |
| Ison 2018 | Ison, G., et al., Clin. Cancer | 10.1158/1078-0432.CCR-18-0042 |
| | Res. 2018, 24, 4066-4071 | |
| Janatová 2003 | Janatová M, et al., Neoplasma, | PMID: 12937835 |
| | 50(4), 246-50 (2003) | |
| Jancárková 2003 | Jancárková, N, Ceska Gynekol., | PMID: 12708108 |
| | 68(1), 11-6 (2003) | |
| Jasin 2002 | Jasin M., Oncogene, 21(58), 8981- | 10.1038/sj.onc.1206176 |
| | 93 (2002) | |
| Jo 2021 | Jo U, et al., Mol Cancer Ther 2021; | 10.1158/1535-7163.MCT-20-1026 |
| | 20: 1431-1441 | |
| Johannes 2021 | Johannes, J W, et al., J Med Chem | 10.1021/acs.jmedchem.1c01012 |
| | 2021, 64, 14498-14512 | |
| Khanna and | Khanna K K and Jackson S P, Nat. | 10.1038/85798 |
| Jackson 2001 | Genet. 27(3): 247-254 (2001) | |
| Kim 2015 | Kim, G., et al., Clin. Cancer | 10.1158/1078-0432.CCR-15-0887 |
| | Res. 2015, 21, 4257-4261 | |
| Kim 2021 | Kim, H., et al Nat Commun 11, 3726 | 10.1038/s41467-020-17127-2 |
| | (2020) | |
| Knegtel 2019 | Knegtel R et al., J Med Chem, 2019, | 10.1021/acs.jmedchem.9b00426 |
| | 62, 5547-5461 | |
| Koul 2021 | Koul, D., et al., Neuro-Oncology, | 10.1093/neuonc/noab196.317 |
| | Volume 23, Issue Supplement 6, | |
| | November 2021, Page vi81 | |
| Lee 2020 | Lee E and Matulonis U, Cancers | 10.3390/cancers12082054 |
| | 2020, 12, 2054 | |
| Lloyd 2020 | Lloyd R L, et al., Oncogene | 10.1038/s41388-020-1328-y |
| | 2020; 39: 4869-4883 | |
| Mahdi 2021 | Mahdi H, et al., JCO Precis Oncol | 10.1200/po.20.00439 |
| | 2021; 5: 1432-42 | |
| Montagnoli 2018 | Montagnoli A, et al. Abstract 4843: | 10.1158/1538-7445.AM2018-4843 |
| | Cancer Res. 2018; 78 (13 Suppl): | |
| | 4843-4843 | |
| Murai 2016 | Murai J, et al., Oncotarget | 10.18632/oncotarget.12266 |
| | 2016; 7: 76534-50 | |
| Neuhausen and | Neuhausen S L and Ostrander E A, | 10.1089/gte.1997.1.75 |
| Ostrander 1992 | Genet. Test, 1, 75-83 (1992) | |
| Papeo 2015 | Papeo G, et al., J Med Chem, 2015, | 10.1021/acs.jmedchem.5b00680 |
| | 58, 6875-6898 | |
| Papeo 2019 | Papeo G, et al., ACS Med Chem Lett | 10.1021/acsmedchemlett.8b00569 |
| | 2019, 10, 534-538 | |
| Patel 2022 | Patel, M, et al., AACR 2022 poster | |
| | OPO.ET05.01, #LB520 | |
| Prados-Carvajal | Prados-Carvajal R, et al., Cancers | 10.3390/cancers14010044 |
| 2021 | (Basel) 2021; 14(1): 44 | |
| Roulston 2022 | Roulston A, et al., Mol Cancer Ther | 10.1158/1535-7163.MCT-21-0615 |
| | (2022) 21(2): 245-256 | |
| Schoonen 2019 | Schoonen P, et al., Molecular | 10.1002/1878-0261.12573 |
| | Oncology, 13 (2019) 2422-2440 | |
| Shah 2021 | Shah P D, et al., Gynecol Oncol. | 10.1016/j.ygyno.2021.08.024 |
| | 2021 November; 163(2): 246-253 | |
| Tutt 2002 | Tutt, et al., Trends Mol Med., 8(12), | 10.1016/s1471-4914(02)02434-6 |
| | 571-576, (2002) | |
| Tutt 2022 | Tutt A, et al., Annals of Oncology | 10.1016/annonc/annonc894 |
| | (2022) 33 (suppl_3): S194-S223 | |
| | and www.medpagetoday.com/ | |
| | meetingcoverage/esmobreastcancer/ | |
| | 98573 | |
| Wang 2020 | Wang J., et al., Journal of Clinical | 10.1200/JCO.2020.38.15_suppl.e15642 |
| | Oncology 38, no. 15_suppl e15642 | |
| Wengner 2020 | Wengner A, et al., Molecular Caner | 10.1158/1535-7163.MCT-19-0019 |
| | Therapeutics 2020, 26-38 | |
| Wilson 2022 | Wilson Z, et al., Cancer Res 2022 82 | 10.1158/0008-5472.CAN-21-2997 |
| | (6): 1140-1152 | |
| Wood 2001 | Wood, et al., Science, 291, 1284- | 10.1126/science.1056154 |
| | 1289 (2001) | |
| Yazinski 2017 | Yazinski S A, et al., Genes Dev | 10.1101/gad.290957.116 |
| | 2017; 31: 318-32 | |
| Yuwen 2022 | Yuwen, H, et al., AACR 2022 poster | |
| | 2604 | |
| Zenke 2019 | Zenke F, et al., Cancer Res (2019) | 10.1158/1538-7445.AM2019-369 |
| | 79 (13_Supplement): 369 | |

1. A method of treating ovarian cancer, breast cancer, gastrointestinal cancer, lung cancer, brain cancer, or prostate cancer in a subject in need thereof, comprising administering to the subject a first amount of a selective PARP1 inhibitor or a pharmaceutically acceptable salt thereof, and a second amount of an ATR inhibitor or a pharmaceutically acceptable salt thereof, wherein the first amount and the second amount together comprise a therapeutically effective amount.

2. The method according to claim 1, wherein the selective PARP1 inhibitor is selected from the group consisting of:
(a) a compound of formula (I): 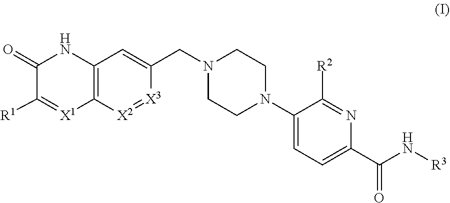
wherein:
X¹ and X² are each independently selected from N and C(H);
X³ is independently selected from N and C(R⁴), wherein R⁴ is H or fluoro;
R¹ is C₁₋₄ alkyl or C₁₋₄ fluoroalkyl;
R² is independently selected from H, halo, C₁₋₄ alkyl, and C₁₋₄ fluoroalkyl; and
R³ is H or C₁₋₄ alkyl;
or a pharmaceutically acceptable salt thereof;
provided that:
when X¹ is N, then X² is C(H) and X³ is C(R⁴);
when X² is N, then X¹ is C(H) and X³ is C(R⁴); and
when X³ is N, then X¹ and X² are both C(H); and
(b) a compound of formula (II): 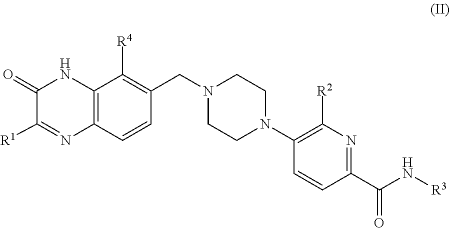
wherein:
R¹ is independently selected from H, C₁₋₄ alkyl, C₁₋₄ fluoroalkyl, and C₁₋₄ alkyloxy;
R² is independently selected from H, halo, C₁₋₄ alkyl, and C₁₋₄ fluoroalkyl; and
R³ is H or C₁₋₄ alkyl;
R⁴ is halo or C₁₋₄ alkyl;
or a pharmaceutically acceptable salt thereof.

3. The method according to claim 1, wherein the selective PARP1 inhibitor is selected from:
(a) 5-{4-[(7-ethyl-6-oxo-5,6-dihydro-1,5-naphthyridin-3-yl)methyl]piperazin-1-yl}-N-methylpyridine-2-carboxamide, or a pharmaceutically acceptable salt thereof; and
(b) 6-fluoro-5-[4-[(5-fluoro-2-methyl-3-oxo-4H-quinoxalin-6-yl)methyl]piperazin-1-yl]-N-methylpyridine-2-carboxamide, or a pharmaceutically acceptable salt thereof.

4. The method according to claim 1, wherein the selective PARP1 inhibitor is 5-{4-[(7-ethyl-6-oxo-5,6-dihydro-1,5-naphthyridin-3-yl)methyl]piperazin-1-yl}-N-methylpyridine-2-carboxamide.

5. The method according to claim 1, wherein the selective PARP1 inhibitor is 6-fluoro-5-[4-[(5-fluoro-2-methyl-3-oxo-4H-quinoxalin-6-yl)methyl]piperazin-1-yl]-N-methylpyridine-2-carboxamide.

6. The method according to claim 1, wherein the ATR inhibitor is selected from the group consisting of:
(a) ceralasertib;
(b) berzosertib;
(c) elimusertib;
(d) VE-821;
(e) gartisertib;
(f) camonsertib;
(g) AZ20;
(h) ATRN-119;
(i) ART-0380;
(j) IMP-9064;
(k) SC-0245;
(l) ATG-018; and
(m) LR-02,
or a pharmaceutically acceptable salt thereof.

7. The method according to claim 6, wherein the ATR inhibitor is ceralasertib.

8. The method according to claim 1, wherein the ovarian cancer is selected from the group consisting of:
(a) advanced epithelial ovarian cancer;
(b) high-grade serous ovarian cancer;
(c) high-grade endometrioid ovarian cancer;
(d) epithelial ovarian cancer comprising a gBRCA1 or a gBRCA2 mutation; and
(e) platinum-sensitive relapsed ovarian cancer, following treatment with a PARP inhibitor.

9. The method according to claim 1, wherein the ovarian cancer is platinum-sensitive relapsed ovarian cancer, following treatment with a PARP inhibitor.

10. The method according to claim 1, wherein the breast cancer is selected from the group consisting of:
(a) deleterious or suspected deleterious gBRCAm, HER2-negative metastatic breast cancer;
(b) deleterious or suspected deleterious gBRCAm, HER2-negative metastatic breast cancer that has been treated with chemotherapy in the neoadjuvant, adjuvant, or metastatic setting;
(c) deleterious or suspected deleterious gBRCAm, HER2-negative, hormone receptor (HR)-positive breast cancer that has been treated with chemotherapy in the neoadjuvant, adjuvant or metastatic setting and has been treated with a prior endocrine therapy or been considered inappropriate for endocrine therapy; and
(d) triple negative breast cancer.

11. The method according to claim 1, wherein the gastrointestinal cancer is selected from the group consisting of:
(a) gastric cancer;
(b) colorectal cancer;
(c) stomach cancer;
(d) liver cancer;
(e) gallbladder cancer;
(f) anal cancer;
(g) pancreatic adenocarcinoma;
(h) deleterious or suspected deleterious gBRCAm pancreatic adenocarcinoma; and
(i) deleterious or suspected deleterious gBRCAm pancreatic adenocarcinoma and the disease has not progressed on at least 16 weeks for a first-line platinum-based chemotherapy regimen.

12. The method according to claim 1, wherein the lung cancer is selected from the group consisting of:
(a) small cell lung cancer; and
(b) non-small cell lung cancer.

13. The method according to claim 1, wherein the brain cancer is selected from the group consisting of:
(a) glioma; and
(b) glioblastoma.

14. The method according to claim 1, wherein the prostate cancer is selected from the group consisting of:
(a) metastatic prostate cancer;
(b) hormone sensitive prostate cancer;
(c) castrate resistant prostate cancer;
(d) metastatic hormone sensitive prostate cancer; and
(e) metastatic castrate resistant prostate cancer.

15.-44. (canceled)

45. A pharmaceutical product comprising i) a selective PARP1 inhibitor or a pharmaceutically acceptable salt thereof, and ii) an ATR inhibitor or a pharmaceutically acceptable salt thereof.

46. A kit comprising: a first pharmaceutical composition comprising a selective PARP1 inhibitor, or a pharmaceutically acceptable salt thereof; a second pharmaceutical composition comprising an ATR inhibitor, or a pharmaceutically acceptable salt thereof; and instructions for using the first and second pharmaceutical compositions in combination.
